(19) United States
(12) Patent Application Publication
(10) Pub. No.: US 2015/0272860 A1
(43) Pub. Date: Oct. 1, 2015

(54) HAIR CARE PRODUCTS WITH SELECTED AMINO ACIDS AND/OR SELECTED OLIGOPEPTIDES AND/OR SELECTED CATIONIC PROTEIN HYDROLYZATES AND SILICONES CONTAINING SUGAR STRUCTURES

(71) Applicant: Henkel AG & Co. KGaA, Duesseldorf (DE)

(72) Inventors: Manuela Mette, Kleinfeld (DE); Christa Hartwich, Elmshorn (DE); Joerg Kahre, Leichlingen (DE)

(21) Appl. No.: 14/735,800

(22) Filed: Jun. 10, 2015

Related U.S. Application Data
(63) Continuation of application No. PCT/EP2013/074213, filed on Nov. 20, 2013.

(30) Foreign Application Priority Data
Dec. 11, 2012 (DE) 10 2012 222 768.7

(51) Int. Cl. A61K 8/894; A61K 8/44; A61Q 5/02; A61K 8/49; A61K 8/81; A61Q 5/00; A61K 8/65; A61K 8/46

(52) U.S. Cl. CPC A61K 8/894; A61K 8/65; A61K 8/44; A61K 8/442; A61K 8/466; A61K 8/4946; A61K 8/8182; A61Q 5/00; A61Q 5/02; A61K 2800/591; A61K 2800/594

(57) ABSTRACT

Hair treatment agents include selected amino acids and/or selected oligopeptides and/or selected cationic protein hydrolysates and silicones that include sugar structures.

### FIELD OF THE INVENTION

The present invention generally relates to hair treatment agents that include selected amino acids and/or selected oligopeptides and/or selected cationic protein hydrolysates and silicones that include sugar structures.

### BACKGROUND OF THE INVENTION

There is a need for further improving hair care products and providing them with further advantageous properties. The aim in particular is to provide a care complex which ideally may also be used in conjunction with oxidizing agents and surfactant agents.

Environmental influences and oxidative hair treatments frequently result in less satisfactory combability of dry and wet hair. In addition, the luster and the moisture balance are adversely affected by the attacked outer structure of the keratinic fibers. Another consequence of repeated treatments of keratinic fibers with surfactant agents and/or oxidative agents is intense greasiness of the keratinic fibers and a pronounced tendency toward increased formation of dandruff.

It is therefore desirable to reduce the side effects of environmental influences and of oxidative and surfactant hair treatments, preferably during the oxidative or surfactant hair treatment, but also after the oxidative or surfactant hair treatment, without reducing the effectiveness of the oxidative or surfactant cosmetic agent, in particular with regard to color intensity, color fastness, lightening power, or wave effect, and to prevent greasiness of the keratinic fibers and the increased formation of dandruff. It is further desirable to combine the oxidative treatment of keratin-containing fibers, in particular human hair, with the application of effective protection of fibers from environmental influences, for example UV protection, also in the form of a 2-in-1 product, in one application step.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Cosmetic composition that includes in a cosmetically acceptable carrier, based on the weight of the overall composition, at least one selected amino acid and/or at least one selected oligopeptide and/or at least one selected cationic protein hydrolysate in a total quantity of 0.01 to 10.0% by weight, and at least one sugar structure-containing silicone of the following formula

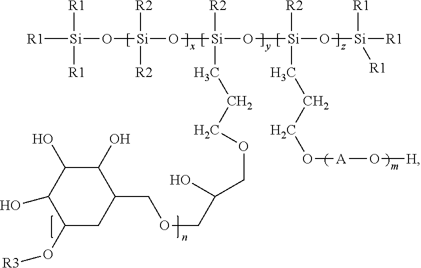

in which the radicals R1, R2, and R3 independently stand for a methyl, ethyl, propyl, isopropyl, hydroxy, methoxy, or ethoxy group, x, y, and z in each case stand for an integer from 1 to 1000, n and m each independently stand for an integer from 1 to 100, in a total quantity of 0.01 to 5.0% by weight.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that the above-described desirable features are achieved in a very satisfactory manner by a hair treatment agent which includes an active substance complex having as essential ingredients at least one selected amino acid and/or one selected protein hydrolysate and/or selected cationic protein hydrolysates and one silicone that includes sugar structures.

Hair treatment agents that include this active substance complex result in improved finish, enhanced luster, improved moisture balance, protection from oxidative damage, prevention of greasiness of the keratinic fibers, increased wash resistance of dyed keratinic fibers, in particular human hair, and retarded formation of dandruff.

A first subject matter of the present invention therefore relates to a hair treatment agent that includes in a suitable cosmetic carrier, in each case based on the overall composition of the agent,
a) at least one selected amino acid and/or at least one selected oligopeptide and/or at least one selected cationic protein hydrolysate in a total quantity of 0.01 to 10.0% by weight,
b) at least one sugar-containing silicone of formula

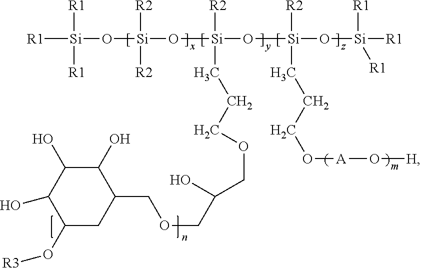

in which the radicals R1, R2, and R3 independently stand for a methyl, ethyl, propyl, isopropyl, hydroxy, methoxy, or ethoxy group, x, y, and z in each case stand for an integer from 1 to 1000, n and m each independently stand for an integer from 1 to 100,
in a total quantity of 0.01 to 5.0% by weight.

Use of this combination results in surprisingly good properties of the treated hair, in particular improved combability, enhanced luster, and improved elasticity, as well as greatly increased wash resistance of dyed hair and longer durability, while at the same time providing better shaping capability for perming processes such as water wave and permanent wave.

In the sense of the present invention, hair treatment agents are understood to mean, for example, hair shampoos, hair conditioners, conditioning shampoos, hair rinses, hair masks, hair packs, hair tonics, hair dye shampoos, or combinations thereof. In particular, the hair conditioning compositions such as hair rinses, hair masks, hair packs, hair oils, and hair lotions, as well as leave-on products, i.e., products that remain on the hair until the next hair washing, and also rinse-off products, i.e., products that are to be rinsed out a few seconds to several hours after application, are understood as the hair treatment agents according to the invention.

According to the invention, combability is understood to mean the combability of wet fibers as well as the combability of dry fibers.

“Hold” is defined as the tactility of a collection of fibers; the parameters of volume and smoothness are felt and assessed by sensory means by those skilled in the art.

Shaping is understood to mean the capability of imparting a change in shape to a collection of previously treated keratin-containing fibers, in particular human hair. In hair cosmetics, this is also referred to as stylability.

In the sense of the invention, restructuring is understood to mean a reduction in the damage to keratinic fibers resulting from various influences. Restoration of the natural strength plays an important role here. Restructured fibers are characterized by enhanced luster, improved hold, and better combability. In addition, they have greater strength and elasticity. Moreover, successful restructuring may be physically demonstrated as an increase in the melting point in comparison to the damaged fiber. The higher the melting point of the hair, the stronger the structure of the fiber.

In the sense of the invention, wash fastness is understood to mean the retention of the original coloring with regard to shade and/or intensity when the keratinic fiber is exposed to the repeated effects from aqueous agents, in particular surfactant-containing agents such as shampoos.

In addition, the compositions according to the invention that include the active substance complex according to the invention are characterized by a greatly improved condition of the keratinic fibers with regard to the moisture balance of the keratinic fibers. Furthermore, the active substance complex according to the invention results in considerable protection of the keratinic fibers from the effects of heat, for example during blow drying of keratinic fibers. The protection of the surface of keratinic fibers from the effects of heat is of major importance in particular when straightening irons or hair dryers are used. Lastly, it has surprisingly been found that the compositions according to the invention result in greatly retarded resoiling of the keratinic fibers.

An aqueous cosmetic carrier includes at least 50% by weight water.

In the sense of the invention, aqueous-alcoholic cosmetic carriers are understood to mean aqueous solutions that include 3 to 70% by weight of a C₁-C₆ alcohol, in particular methanol, ethanol, or propanol, isopropanol, butanol, isobutanol, tert-butanol, n-pentanol, isopentanols, n-hexanol, isohexanols, glycol, glycerin, 1,2-pentanediol, 1,5-pentanediol, 1,2-hexanediol, or 1,6-hexanediol. The agents according to the invention may additionally include further organic solvents, for example methoxybutanol, benzyl alcohol, ethyl diglycol, or 1,2-propylene glycol. All water-soluble organic solvents are preferred, with water being particularly preferred.

The first ingredient a) according to the invention is at least one selected amino acid and/or at least one selected oligopeptide and/or at least one selected cationic protein hydrolysate. In the present patent application, the term “amino acid” is also understood to mean a structure which includes only one permanent cationic group, for example choline, in the molecule. In addition, this term is also understood to mean substances such as carnitine or taurine, since these substances, the same as amino acids, naturally occur in biological systems, and in many cases behave like amino acids.

Amino acids according to the invention are selected from alanine, arginine, asparagine, aspartic acid, cysteine, cystine, glutamic acid, glutamine, glycine, histidine, hydroxylysine, hydroxyproline, isoleucine, leucine, lysine, methionine, phenylalanine, proline, serine, threonine, thyroxine, tryptophan, tyrosine, valine, betaine, ornithine, 1,1-dimethyl proline, hercynine (Nα,Nα,Nα-trimethyl-L-histidinium betaine), ergothioneine (thioneine, 2-mercapto-Nα,Nα,Nα-trimethyl-L-histidinium betaine), carnitine, taurine, and choline, and the mixtures thereof. All types of isomers, such as diastereomers, enantiomers, cis/trans isomers, optical isomers, conformational isomers, and racemates may be used according to the invention.

Alanine, arginine, asparagine, glutamic acid, glutamine, glycine, histidine, hydroxylysine, hydroxyproline, isoleucine, leucine, lysine, proline, serine, betaine, ornithine, 1,1-dimethyl proline, carnitine, taurine, choline, and the mixtures thereof are particularly preferably used.

Arginine, glutamine, glycine, histidine, lysine, proline, serine, betaine, carnitine, taurine, and the mixtures thereof are very particularly preferably used.

Arginine, glutamine, histidine, lysine, carnitine, and taurine, and the mixtures thereof are extremely preferably used.

Arginine, glutamine, carnitine, and taurine and the mixtures of: arginine and taurine; glutamine and taurine; glutamine and carnitine; arginine and glutamine; carnitine and taurine, and the mixtures of: arginine, carnitine, and taurine; glutamine, carnitine, and taurine are most preferred.

Oligopeptides selected according to the invention are usable as an alternative to and/or together with the above-described amino acids. Within the meaning of the present patent application, oligopeptides are condensation products of amino acids, comprising at least 3 and no more than 25 amino acids, linked by peptide bonds in the manner of an acid amide. In hair treatment agents preferred according to the invention, the oligopeptide comprises 5 to 15 amino acids, preferably 6 to 13 amino acids, particularly preferably 7 to 12 amino acids, and in particular 8, 9, or 10 amino acids.

Depending on whether further amino acids are bound to the Glu-Glu-Glu sequence, and the type of these amino acids, the molar mass of the oligopeptide included in the agents according to the invention may vary. Hair treatment agents preferred according to the invention are characterized in that the oligopeptide has a molar mass of 650 to 3000 Da, preferably 750 to 2500 Da, particularly preferably 850 to 2000 Da, and in particular 1000 to 1600 Da.

As can be seen from the preferred number of amino acids in the oligopeptides and the preferred molar mass range, oligopeptides are preferably used which do not consist solely of the three glutamic acids, but, rather, which include further amino acids bound to this sequence. These further amino acids are preferably selected from specific amino acids, whereas certain other representatives are less preferred according to the invention.

Thus, it is preferred that the oligopeptides used in the agents according to the invention include no methionine. It is further preferred that the oligopeptides used in the agents according to the invention include no cysteine and/or cystine. It is further preferred that the oligopeptides used in the agents according to the invention include no aspartic acid and/or asparagine. It is further preferred that the oligopeptides used in the agents according to the invention include no serine and/or threonine. In contrast, it is preferred that the oligopeptides used in the agents according to the invention include tyrosine. It is further preferred that the oligopeptides used in the agents according to the invention include leucine. It is further preferred that the oligopeptides used in the agents according to the invention include isoleucine. It is further preferred that the oligopeptides used in the agents according to the invention include arginine. It is further preferred that the oligopeptides used in the agents according to the invention include valine.

Particularly preferred oligopeptides and amino acid sequences included in the preferred oligopeptides are described below:

One particularly preferred oligopeptide additionally includes tyrosine, which is preferably bound via its acid function to the Glu-Glu-Glu sequence. Hair treatment agents preferred according to the invention are therefore characterized in that the oligopeptide included therein comprises at least one amino acid sequence Tyr-Glu-Glu-Glu, wherein the amino group may be present in free or protonated form, and the carboxy groups may be present in free or deprotonated form.

Another particularly preferred oligopeptide additionally includes isoleucine, which is preferably bound via its amino function to the Glu-Glu-Glu sequence. Hair treatment agents preferred according to the invention are therefore characterized in that the oligopeptide included therein comprises at least one amino acid sequence Glu-Glu-Glu-Ile, wherein the amino group may be present in free or protonated form, and the carboxy groups may be present in free or deprotonated form.

Oligopeptides which comprise both of the above-mentioned amino acids (tyrosine and isoleucine) are preferred according to the invention. Hair treatment agents according to the invention are particularly preferred in which the oligopeptide included therein comprises at least one amino acid sequence Tyr-Glu-Glu-Glu-Ile, wherein the amino group may be present in free or protonated form, and the carboxy groups may be present in free or deprotonated form.

Further preferred oligopeptides additionally include arginine, which is preferably present bound to isoleucine. An extremely preferred oligopeptide is commercially available under the trade name ProSina® from Croda.

The hair treatment agents according to the invention include the selected amino acids and/or the selected oligopeptides as described above in a total quantity of 0.0001 to 10.0% by weight, particularly preferably 0.0001 to 7.0% by weight, very particularly 0.0001 to 5.0% by weight, based on the overall agent.

Selected cationic protein hydrolysates are a further component according to the invention which may be used as the selected amino acids and/or selected oligopeptides. Cationic keratin hydrolysates are very particularly preferably used according to the invention. Among these, a cationic keratin hydrolysate is preferred which corresponds to formula (I)

R′—X—R″ (I),

in which
R′ stands for a straight-chain or branched, saturated or unsaturated hydrocarbon radical having 11 to 24 carbon atoms,
R″ means a protein, a peptide, or a protein hydrolysate,
X stands for C(O)O— or —N⁺(R^{III}₂)R^{IV}— or —N(R^{III})R^{IV}— or —C(O)—N(R^{V})R^{VI}—,
R^{III} means —(CH₂)ₓ—CH₃, where x=0-22, and
R^{IV} means —CH₂—CH(OH)—CH₂— or —(CH₂)ₓ, where x=0-22;
R^{V} and R^{VI} independently stand for —H or —(CH₂)ₓ—CH₃, where x=0-22;
   with the condition that R″ stands for keratin or a keratin hydrolysate.

The radical R′ most preferably stands for a lauryl group, and X stands for —N⁺(R^{III}₂)R^{IV}—, particularly preferably with R^{III} standing for —CH₃ and R^{IV} standing for —CH₂—CH(OH)—CH₂—, and R″ most preferably stands for a hydrolysate obtained from the cortex and/or the cuticle of keratinic fibers. The product with the INCI name Laurdimonium Hydroxypropyl Hydrolyzed Keratin is most preferred.

The cationic keratin hydrolysates according to the invention are included in the compositions according to the invention in a quantity of 0.01 to 10.0% by weight, preferably 0.01 to 7.5% by weight, particularly preferably 0.1 to 5.0% by weight, based on the overall composition.

The second mandatory component b) of the active substance complex is a sugar structure-containing silicone of the following formula:

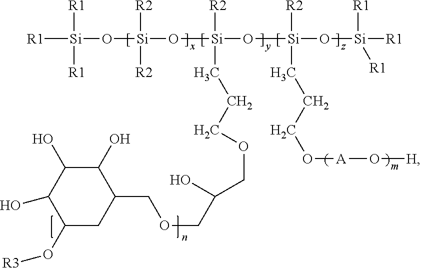

in which the radicals R1, R2, and R3 independently stand for a methyl, ethyl, propyl, isopropyl, hydroxy, methoxy, or ethoxy group, x, y, and z in each case stand for an integer from 1 to 1000, and n and m in each case independently stand for an integer from 1 to 100. The radicals R1 preferably independently stand for methyl, hydroxy, or methoxy, particularly preferably for methyl. The radicals R2 and R3 preferably independently stand for methyl, ethyl, propyl, or isopropyl, particularly preferably for methyl. x, y, and z in each case preferably independently stand for an integer from 1 to 500, and particularly preferably from 1 to 200. n and m preferably independently stand for integers from 1 to 50. m particularly preferably stands for an integer from 1 to 20, most preferably from 1 to 15, and in particular for the numbers 2, 3, 4, 5, 6, 7, 8, 9, 10, 11, 12, 13, 14, 15. n particularly preferably stands for an integer from 1 to 20, most preferably from 1 to 10, and in particular for a number 1, 2, 3, 4, 5, 6, 7, 8, 9, 10. A stands for a group selected from —CH₂CH₂—, —CH₂CH₂CH₂—, or —CH₂CH₂CH₂CH₂— or mixtures of these groups, and is preferably selected from —CH₂CH₂—, —CH₂CH₂CH₂—, and the mixtures thereof, most preferably —CH₂CH₂—.

An extremely preferred silicone of the above-described formula corresponds to the formula illustrated below:

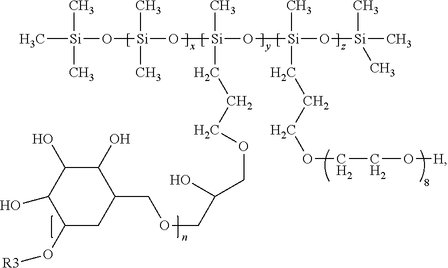

in which R3 has the meaning described above, and most preferably stands for methyl, and x, y, and z, and n and m have the meanings described above.

Such products are commercially available under the trade name PolySuga Sil from Colonial. One particularly preferred aminosilicone is obtainable under the trade names Poly Suga® Sil C-35P and/or Poly Suga® Sil C-800P. An extremely preferred silicone that includes sugar structures is obtainable under the INCI name PEG-8 PG-Coco-Glucoside Dimethicone.

These sugar-containing silicone polymers are included in the compositions according to the invention in quantities of 0.01 to 5% by weight, preferably in quantities of 0.05 to 5% by weight, and very particularly preferably in quantities of 0.1 to 5% by weight. The very best results are obtained using quantities of 0.1 to 2.5% by weight, in each case based on the overall composition of the particular agent.

According to the invention, it is preferred that the compositions according to the invention also include at least one quaternary compound. The effectiveness of the agent according to the invention is thus even further increased, and the stability of the composition is significantly improved.

Quaternary ammonium compounds are in principle monomeric, cationic, or amphoteric ammonium compounds, monomeric amines, aminoamides, polymeric cationic ammonium compounds, and polymeric amphoteric ammonium compounds. Of this plurality of possible quaternary ammonium compounds, the following groups have proven to be particularly suitable, and, taken separately, are each used in a quantity of 0.1 to 15.0% by weight. The quantity does not fall below or exceed this value, even when a mixture of different compounds of the quaternary ammonium compounds is used.

Cationic surfactants of formula (Tkat1-1) constitute the first group of cationic surfactants.

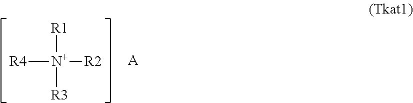

In formula (Tkat1), R1, R2, R3, and R4 in each case independently stand for hydrogen, a methyl group, a phenyl group, a benzyl group, or for a saturated, branched or unbranched alkyl radical which has a chain length of 8 to 30 carbon atoms and which may optionally be substituted with one or more hydroxy groups. A stands for a physiologically acceptable anion, for example halides such as chloride or bromide, and methosulfates.

Examples of compounds of formula (Tkat1) are lauryl trimethylammonium chloride, cetyl trimethylammonium chloride, cetyl trimethylammonium bromide, cetyl trimethylammonium methosulfate, dicetyl dimethylammonium chloride, tricetyl methylammonium chloride, stearyl trimethylammonium chloride, distearyl dimethylammonium chloride, lauryl dimethylbenzylammonium chloride, behenyl trimethylammonium chloride, behenyl trimethylammonium bromide, and behenyl trimethylammonium methosulfate.

Esterquats according to formula (Tkat2) constitute a preferred group

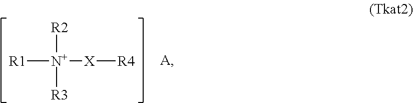

in which the radicals R1, R2, and R3 in each case are independent of one another, and may be the same or different. The radicals R1, R2, and R3 mean:
a branched or unbranched alkyl radical which has 1 to 4 carbon atoms, and which may include at least one hydroxyl group, or
a saturated or unsaturated, branched or unbranched, or cyclic saturated or unsaturated alkyl radical which has 6 to 30 carbon atoms, and which may include at least one hydroxyl group, or
an aryl or alkaryl radical, for example phenyl or benzyl,
the radical (—X—R4), with the condition that no more than two of the radicals R1, R2, or R3 may stand for this radical:
   The radical —(X—R4) is included at least 1 to 3 times.
   In the formula, X stands for:
1) —(CH₂)ₙ—, where n=1 to 20, preferably n=1 to 10, and particularly preferably n=1 to 5, or
2) —(CH₂—CHR5-O)ₙ—, where n=1 to 200, preferably 1 to 100, particularly preferably 1 to 50, and very particularly preferably 1 to 20, where R5 means hydrogen, methyl, or ethyl,
3) a hydroxyalkyl group which has 1 to 4 carbon atoms and which may be branched or unbranched, and which includes at least one and no more than three hydroxy groups. Examples are: —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH, and hydroxybutyl radicals,
   and R4 stands for:
1) R6-O—CO—, where R6 is a saturated or unsaturated, branched or unbranched, or a cyclic saturated or unsaturated alkyl radical which has 6 to 30 carbon atoms and which may include at least one hydroxy group, and which optionally may also be oxethylated with 1 to 100 ethylene oxide units and/or 1 to 100 propylene oxide units, or
2) R7-CO—, where R7 is a saturated or unsaturated, branched or unbranched, or a cyclic saturated or unsaturated alkyl radical which has 6 to 30 carbon atoms and which may include at least one hydroxy group, and which optionally may also be oxethylated with 1 to 100 ethylene oxide units and/or 1 to 100 propylene oxide units,
   and A stands for a physiologically acceptable organic or inorganic anion, and at this point is defined as a representative also for all structures described below. The anion of all described cationic compounds is selected from the halide ions fluoride, chloride, bromide, and iodide, and sulfates of general formula RSO₃⁻, where R has the meaning of saturated or unsaturated alkyl radicals having 1 to 4 carbon atoms, or anionic radicals of organic acids such as maleate, fumarate, oxalate, tartrate, citrate, lactate, or acetate.

Such products are marketed under the trademarks Rewoquat®, Stepantex®, Dehyquart®, Armocare®, and Akypoquat®, for example. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90, and Akypoquat® 131 are examples of these esterquats.

Further compounds of formula (Tkat1-2) that are particularly preferred according to the invention are those of formula (Tkat2.1), the cationic betaine esters.

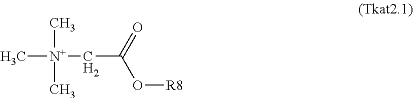

The meaning of R8 corresponds to that of R7.

Esterquats with the trade names Armocare® VGH-70, Dehyquart® F-75, Dehyquart® L80, Stepantex® VS 90, and Akypoquat® 131 are particularly preferred.

In preferred agents according to the invention, cationic surfactants of formula (I) are used within fairly narrow quantity ranges, so that preferred hair treatment agents according to the invention are characterized in that they include 0.1 to 15% by weight, preferably 0.5 to 10% by weight, more preferably 1 to 10% by weight, even more preferably 1.5 to 10% by weight, and in particular 2 to 5% by weight of at least one compound of general formula (I)

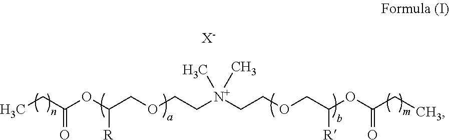

in which
n and m independently stand for integers between 5 and 40, with the condition that n+m≧38; n=m is particularly preferred; n=m=20 is most preferred.
a and b independently stand for integers between 1 and 10; in particular independently stand for 1, 2, 3, 4, or 5; the equation a+2≧b≧a−2 preferably applies, and it is most preferred that a=b=3.
R and R′ are independently selected from —H and —CH₃; R=R′ preferably applies, so that preferably either PEG or PPG diesterquats are used; R=R′═—CH₃ very particularly preferably applies.
X⁻ is a physiologically acceptable anion, a halide such as chloride, bromide, or iodide, or toluenesulfonate, methosulfate, and the like, and is particularly preferably methosulfate.

In particular when one of the compounds of formula (I) as described above is used, it has been shown that the care effects of the agents according to the invention are further increased, and in particular the stability of the agents may be further improved, when the agents include certain acylated diamines in addition to the compound(s) of formula (I).

Hair treatment agents which are preferred according to the invention are therefore characterized in that they additionally include 0.1 to 10% by weight of at least one compound of formula (II)

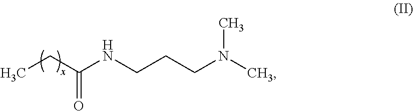

in which x stands for 18, 19, 20, 21, 22, 23, or 24.

Compounds of formula (II) where n=20 are particularly preferred. Extremely preferred agents according to the invention are characterized in that they include a compound of formula (I), always together with a compound of general formula (II).

Quaternary imidazoline compounds are a further group. The structure of these compounds is shown in formula (Tkat2) below.

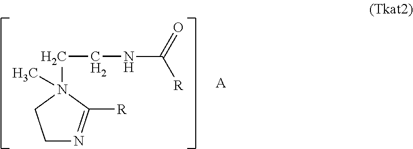

The radicals R in each case independently stand for a saturated or unsaturated, linear or branched hydrocarbon radical having a chain length of 8 to 30 carbon atoms. The preferred compounds of formula (Tkat2) in each case include the same hydrocarbon radical for R. The chain length of the radicals R is preferably 12 to 21 carbon atoms. A stands for an anion as described above. Examples which particularly conform to the invention are obtainable under the INCI names Quaternium-27, Quaternium-72, Quaternium-83, and Quaternium-91, for example. Quaternium-91 is most preferred according to the invention.

In one particularly preferred embodiment of the invention, the agents according to the invention also include at least one amine and/or cationized amine, in particular an amidoamine and/or a cationized amidoamine, having the following structural formula:

R1-NH—(CH₂)ₙ—N⁺R²R³R₄A (Tkat3),

where R1 is an acyl or alkyl radical which has 6 to 30 C atoms and which may be branched or unbranched, saturated or unsaturated, and wherein the acyl radical and/or the alkyl radical may include at least one OH group, and
R2, R3, and R4 in each case independently stand for
1) hydrogen or
2) an alkyl radical which has 1 to 4 C atoms and which may be the same or different, saturated or unsaturated, and
3) a branched or unbranched hydroxyalkyl group which has 1 to 4 carbon atoms and which includes at least one and no more than three hydroxy groups, for example —CH₂OH, CH₂CH₂OH, —CHOHCHOH, —CH₂CHOHCH₃, —CH(CH₂OH)₂, —COH(CH₂OH)₂, CH₂CHOHCH₂OH, —CH₂CH₂CH₂OH, and hydroxybutyl radicals,
   A means an anion as described above, and
   n means an integer between 1 and 10.

A composition is preferred in which the amine and/or the quaternized amine according to general formula (Tkat3) is/are an amidoamine and/or a quaternized amidoamine, where R1 means a branched or unbranched, saturated or unsaturated acyl radical which has 6 to 30 C atoms and which may include at least one OH group. In this regard, a fatty acid radical obtained from oils and waxes, in particular from natural oils and waxes, is preferred. Suitable examples of such are lanolin, beeswax, or candellila wax.

Also preferred are amidoamines and/or quaternized amidoamines in which R2, R3, and/or R4 in formula (Tkat3) mean a radical according to general formula CH₂CH₂OR5, where R5 may have the meaning of alkyl radicals having 1 to 4 carbon atoms, hydroxyethyl, or hydrogen. The preferred value of n in general formula (Tkat8) is an integer between 2 and 5.

The alkylamidoamines may be present as such, or may also be converted to a quaternary compound in the composition by protonation in an appropriately acidic solution. Cationic alkylamidoamines are preferred according to the invention.

Examples of such commercial products according to the invention are Witcamine® 100, Incromine® BB, Mackine® 401 and other Mackine® types, Adogen® S18V, and as permanent cationic amidoamines: Rewoquat® RTM 50, Empigen® CSC, Swanol® Lanoquat DES-50, Rewoquat® UTM 50, Schercoquat® BAS, Lexquat® AMG-BEO, or Incroquat® Behenyl HE.

A further fatty acid amide according to the invention corresponds to general formula (I)

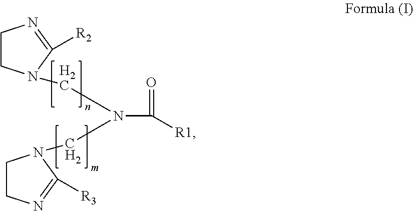

in which R1, R2, and R3 independently stand for a linear branched or unbranched C6 to C30, preferably C8 to C24, more preferably C12 to C22, and most preferably C12 to C18, alkyl or alkenyl group. and R1 to R3 preferably stand for capryl, caprylyl, octyl, nonyl, decanyl, lauryl, myristyl, cetyl, stearyl, isostearyl, oleyl, behenyl, or arachidyl. In addition, it is particularly preferred that R2=R3, and it is most preferred that R1=R2=R3. The letters n and m independently stand for integers from 1 to 10, preferably 2 to 6, and most preferably for 2, 3, and/or 4, with n=m being extremely preferred. It is most preferred that R1=R2=R3 and are selected from capryl, caprylyl, octyl, nonyl, decanyl, lauryl, myristyl, cetyl, stearyl, isostearyl, oleyl, behenyl, or arachidyl, and n=m=2. It is most preferred that R1=R2=R3 and are selected from lauryl, myristyl, cetyl, stearyl, isostearyl, oleyl, behenyl, or arachidyl, among which cetyl, stearyl, isostearyl, oleyl, or behenyl are particularly preferred, and n=m=2. The most preferred compound of formula (I) is that having the INCI name Bis-Ethyl(isostearylimidazoline) Isostearamide. The latter compound is commercially available from Croda under the trade name Keradyn® HH.

The above-mentioned cationic surfactants may be used individually or in any combinations with one another, in quantities of 0.01 to 10% by weight, preferably in quantities of 0.01 to 7.5% by weight, and very particularly preferably in quantities of 0.1 to 5.0% by weight. The very best results are obtained with quantities of 0.1 to 3.0% by weight, in each case based on the overall composition of the particular agent.

Cationic and amphoteric polymers are further quaternary ammonium compounds.

The cationic and/or amphoteric polymers may be homopolymers, copolymers, or polymers based on natural polymers, the quaternary nitrogen groups being included either in the polymer chain or preferably as a substituent of one or more of the monomers. The ammonium group-containing monomers may be copolymerized with noncationic monomers. Suitable cationic monomers are unsaturated, radically polymerizable compounds bearing at least one cationic group, in particular ammonium-substituted vinyl monomers such as trialkylmethacryloxyalkyl ammonium, trialkylacryloxyalkyl ammonium, dialkyldiallyl ammonium, and quaternary vinyl ammonium monomers having cyclic, cationic nitrogen-containing groups such as pyridinium or imidazolium, or quaternary pyrrolidones such as alkyl vinylimidazolium, alkyl vinylpyridinium, or alkyl vinylpyrrolidone salts. The alkyl groups of these monomers are preferably lower alkyl groups such as C1 to C7 alkyl groups, particularly preferably C1 to C3 alkyl groups.

The ammonium group-containing monomers may be copolymerized with noncationic monomers. Examples of suitable comonomers are acrylamide, methacrylamide, alkyl and dialkyl acrylamide, alkyl and dialkyl methacrylamide, alkyl acrylate, alkyl methacrylate, vinyl caprolactone, vinyl caprolactam, vinylpyrrolidone, vinyl esters, for example vinyl acetate; vinyl alcohol, propylene glycol, or ethylene glycol, wherein the alkyl groups of these monomers are preferably C1 to C7 alkyl groups, particularly preferably C1 to C3 alkyl groups.

Of the plurality of these polymers, homopolymers of general formula

—{CH₂—[CR¹COO—(CH₂)ₘN⁺R²R³R⁴]}ₙX⁻

have proven to be particularly effective components of the active substance complex according to the invention,
in which R¹ is —H or —CH₃, R², R³, and R⁴ are independently selected from C1-4 alkyl, alkenyl, or hydroxyalkyl groups, m=1, 2, 3, or 4, and n is a natural number, and
X⁻ is a physiologically acceptable organic or inorganic anion. Of these polymers, preferred according to the invention are those for which at least one of the following conditions is met: R¹ stands for a methyl group, R², R³, and R⁴ stand for methyl groups, and m has the value 2.

Halide ions, sulfate ions, phosphate ions, methosulfate ions, and organic ions such as lactate, citrate, tartrate, and acetate ions are examples of suitable physiologically acceptable counterions X⁻. Methosulfate and halide ions, in particular chloride, are preferred.

Suitable cationic polymers are, for example, copolymers according to formula (Copo) which are preferably included in the hair treatment agents according to the invention in a quantity of 0.001 to 5% by weight, preferably 0.0025 to 2.5% by weight, particularly preferably 0.005 to 1% by weight, more preferably 0.0075 to 0.75% by weight, and in particular 0.01 to 0.5% by weight, based on their weight.

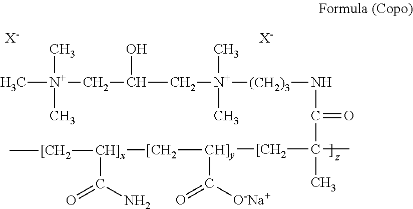

in which:

x+y+z=Q,
Q stands for values from 3 to 55,000, preferably from 10 to 25,000, particularly preferably from 50 to 15,000, more preferably from 100 to 10,000, even more preferably from 500 to 8000, and in particular from 1000 to 5000,
x stands for (0 to 0.5) Q, preferably for (0 to 0.3) Q, and in particular for the values 0, 1, 2, 3, 4, 5, with the value 0 being preferred,
y stands for (0.1 to 0.95) Q, preferably for (0.5 to 0.7) Q, and in particular for values from 1 to 24,000, preferably from 5 to 15,000, particularly preferably from 10 to 10,000 and in particular from 100 to 4800,
z stands for (0.001 to 0.5) Q, preferably for (0.1 to 0.5) Q, and in particular for values from 1 to 12,500, preferably from 2 to 8000, particularly preferably from 3 to 4000, and in particular from 5 to 2000.

Regardless of which of the preferred copolymers of formula (Copo) is used, hair treatment agents according to the invention are preferred which are characterized in that the y:z ratio is 4:1 to 1:2, preferably 4:1 to 1:1.

Regardless of which copolymers are used in the agents according to the invention, hair treatment agents according to the invention are preferred in which the copolymer has a molar mass of 10,000 to 20 million gmol⁻¹, preferably 100,000 to 10 million gmol⁻¹, more preferably 500,000 to 5 million gmol⁻¹, and in particular 1.1 million to 2.2 million gmol⁻¹.

An extremely preferred copolymer having a structure as described above is commercially available under the name Polyquaternium-74.

A particularly suitable homopolymer is optionally crosslinked poly(methacryloyloxyethyltrimethylammonium chloride) having the INCI name Polyquaternium-37. Such products are commercially available under the names Rheocare® CTH (Cosmetic Rheologies) and Synthalen® CR (3V Sigma), for example.

The homopolymer is preferably used in the form of a nonaqueous polymer dispersion. Such polymer dispersions are commercially available under the names Salcare® SC 95 and Salcare® SC 96.

Suitable cationic polymers that are derived from natural polymers are cationic derivatives of polysaccharides, for example cationic derivatives of cellulose, starch, or guar. Chitosan and chitosan derivatives are also suitable. Cationic polysaccharides have the general formula G-O—B—N⁺RₐR_{b}R_{c}A⁻,

where
G is an anhydroglucose radical, for example starch anhydroglucose or cellulose anhydroglucose;
B is a divalent connecting group, for example alkylene, oxyalkylene, polyoxyalkylene, or hydroxyalkylene;
Rₐ, R_{b}, and R_{c} are independently alkyl, aryl, alkylaryl, arylalkyl, alkoxyalkyl. or alkoxyaryl, in each case having up to 18 C atoms, wherein the total number of C atoms in Rₐ, R_{b}, and R_{c} is preferably 20 maximum;
A⁻ is a customary counteranion, preferably chloride.

Cationic, i.e., quaternized, celluloses are available on the market with different degrees of substitution, cationic charge densities, nitrogen contents, and molecular weights. For example, Polyquaternium-67 is marketed under the names Polymer® SL or Polymer® SK (Amerchol). Another extremely preferred cellulose is marketed by Croda under the trade name Mirustyle® CP. This is a trimonium and cocodimonium hydroxyethylcellulose as derivatized cellulose with the INCI name Polyquaternium-72. Polyquaternium-72 may be used in solid form, or predissolved in aqueous solution.

Further cationic celluloses are Polymer JR® 400 (Amerchol, INCI name Polyquaternium-10) and Polymer Quatrisoft® LM-200 (Amerchol, INCI name Polyquaternium-24). Further commercial products are the compounds Celquat® H 100 and Celquat® L 200. Polyquaternium-24, Polyquaternium-67, and Polyquaternium-72 are particularly preferred cationic celluloses.

Suitable cationic guar derivatives are marketed under the trade name Jaguar®, and have the INCI name Guar Hydroxypropyltrimonium Chloride. In addition, particularly suitable cationic guar derivatives are also marketed by Hercules under the name N-Hance®. Further cationic guar derivatives are marketed by Cognis under the name Cosmedia®. A preferred cationic guar derivative is the commercial product AquaCat® from Hercules. This raw material is a predissolved cationic guar derivative. Cationic guar derivatives are preferred according to the invention.

A suitable chitosan is marketed, for example, by Kyowa Oil & Fat, Japan, under the trade name Flonac®. A preferred chitosan salt is chitosonium pyrrolidone carboxylate, which is marketed, for example, by Amerchol, US under the name Kytamer® PC. Further chitosan derivatives are readily commercially available under the trade names Hydagen® CMF, Hydagen® HCMF, and Chitolam® NB/101.

Polymers based on glucose are another group of polymers that may be used according to the invention with excellent results. The following illustration shows such a cationic alkyl oligoglucoside.

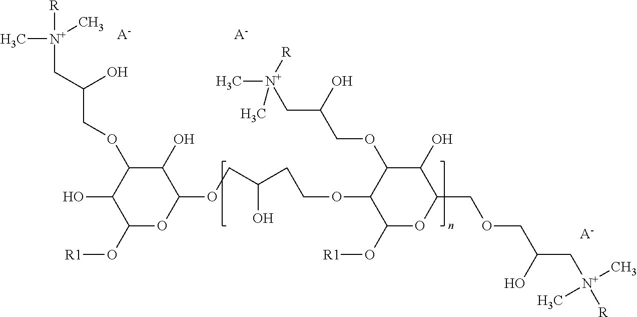

In the formula illustrated above, the radicals R independently stand for a linear or branched C6 to C30 alkyl radical or a linear or branched C6 to C30 alkenyl radical; the radical R preferably stands for a radical R selected from: lauryl, myristyl, cetyl, stearyl, oleyl, behenyl, or arachidyl.

The radicals R1 independently stand for a linear or branched C6 to C30 alkyl radical or a linear or branched C6 to C30 alkenyl radical; the radical R preferably stands for a radical selected from: butyl, capryl, caprylyl, octyl, nonyl, decanyl, lauryl, myristyl, cetyl, stearyl, oleyl, behenyl, or arachidyl. The radicals R1 are particularly preferably the same. The radicals R1 are more preferably selected from technical mixtures of the fatty alcohol cuts of C6/C8 fatty alcohols, C8/C10 fatty alcohols, C10/C12 fatty alcohols, C12/C14 fatty alcohols, C12/C18 fatty alcohols, and most preferably are technical fatty alcohol cuts of plant origin. The counterion for the cationic charge is a physiologically acceptable anion, for example halide, methosulfate, phosphate, citrate, tartrate, etc. The counterion is preferably a halide such as fluoride, chloride, or bromide, or methosulfate. The anion chloride is most preferred.

Particularly preferred examples of cationic alkyl oligoglucosides are the compounds having the INCI names Polyquaternium-77, Polyquaternium-78, Polyquaternium-79, Polyquaternium-80, Polyquaternium-81, and Polyquaternium-82. Cationic alkyl oligoglucosides having the names Polyquaternium-77, Polyquaternium-81, and Polyquaternium-82 are most preferred.

These types of compounds may be obtained, for example, from Colonial Chemical Inc. under the name Poly Suga® Quat.

The cationic alkyl oligoglucosides are used in a total quantity of 0.01 to 10.0% by weight, preferably 0.05 to 5.0% by weight, more preferably 0.1 to 3.0% by weight, and most preferably in quantities of 0.2 to 2.0% by weight, in each case based on the total weight of the composition. Of course, use of a larger number of mixtures of cationic alkyl oligoglucosides is also encompassed by the invention. In this case, it is preferred to simultaneously use a long-chain and a short-chain cationic alkyl oligoglucoside in each case.

Another preferred cationic polymer based on ethanolamine is obtainable. The polymer is commercially available under the name Polyquaternium-71.

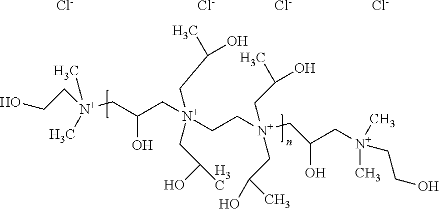

This polymer may be obtained, for example, from Colonial Chemical Inc. under the name Cola® Moist 300 P.

Polyquaternium-71 is used in a total quantity of 0.01 to 10.0% by weight, preferably 0.05 to 5.0% by weight, more preferably 0.1 to 3.0% by weight, and most preferably in quantities of 0.2 to 2.0% by weight, in each case based on the total weight of the composition.

In addition, a cationic alkyl oligoglucoside as shown in the following illustration may particularly preferably be used.

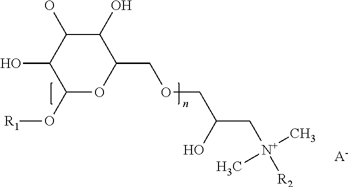

In the formula illustrated above, the radical R2 stands for a linear or branched C6 to C30 alkyl radical or a linear or branched C6 to C30 alkenyl radical; the radical R preferably stands for a radical R selected from: lauryl, myristyl, cetyl, stearyl, oleyl, behenyl, or arachidyl.

The radical R1 stands for a linear or branched C6 to C30 alkyl radical or a linear or branched C6 to C30 alkenyl radical; the radical R1 preferably stands for a radical selected from: butyl, capryl, caprylyl, octyl, nonyl, decanyl, lauryl, myristyl, cetyl, stearyl, oleyl, behenyl, or arachidyl. The radical R1 is more preferably selected from technical mixtures of the fatty alcohol cuts of C6/C8 fatty alcohols, C8/C10 fatty alcohols, C10/C12 fatty alcohols, C12/C14 fatty alcohols, C12/C18 fatty alcohols, and most preferably technical fatty alcohol cuts of plant origin. The Index n stands for a number between 1 and 20, preferably between 1 and 10, more preferably between 1 and 5, and most preferably between 1 and 3. The counterion for the cationic charge, A⁻, is a physiologically acceptable anion, for example halide, methosulfate, phosphate, citrate, tartrate, etc. The counterion is preferably a halide such as fluoride, chloride, or bromide, or methosulfate. The anion chloride is most preferred.

Particularly preferred examples of cationic alkyl oligoglucosides are the compounds having the INCI names Laurdimoniumhydroxypropyl Decylglucosides Chloride, Laurdimoniumhydroxypropyl Laurylglucosides Chloride, Stearyldimoniumhydroxypropyl Decylglucosides Chloride, Stearyldimoniumhydroxypropyl Laurylglucosides Chloride, Stearyldimoniumhydroxypropyl Laurylglucosides Chloride, or Cocoglucosides Hydroxypropyltrimonium Chloride.

Such compounds may be obtained, for example, from Colonial Chemical Inc. under the name Suga® Quat.

The cationic alkyl oligoglucosides are used in a total quantity of 0.01 to 10.0% by weight, preferably 0.05 to 5.0% by weight, more preferably 0.1 to 3.0% by weight, and most preferably in quantities of 0.2 to 2.0% by weight, in each case based on the total weight of the composition. Of course, use of a larger number of mixtures of cationic alkyl oligoglucosides is also encompassed by the invention. In this case, it is preferred to simultaneously use a long-chain and a short-chain cationic alkyl oligoglucoside in each case.

Another preferred cationic polymer includes at least one structural unit of formula (I), at least one structural unit of formula (II), at least one structural unit of formula (III), and at least one structural unit of formula (IV)

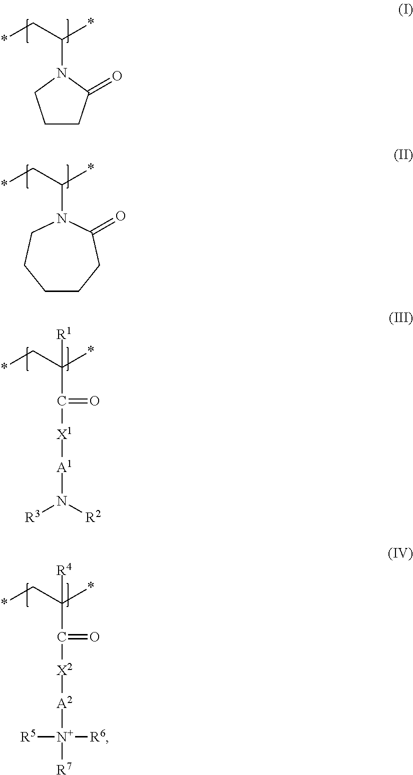

where
R¹ and R⁴ independently stand for a hydrogen atom or a methyl group,
X¹ and X² independently stand for an oxygen atom or an NH group,
A¹ and A² independently stand for an ethane-1,2-diyl, propane-1,3-diyl, or butane-1,4-diyl group,
R², R³, R⁵, and R⁶ independently stand for a (C₁ to C₄) alkyl group, and
R⁷ stands for a (C₈ to C₃₀) alkyl group.

According to the above formulas and all of the formulas below, a chemical bond denoted by the symbol “*” stands for a free valence of the corresponding structural fragment.

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate are used to compensate for the positive polymer charge in the agent according to the invention.

Examples of (C, to C₄) alkyl groups according to the invention are methyl, ethyl, propyl, isopropyl, butyl, sec-butyl, isobutyl, and tert-butyl.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), and docosyl (behenyl).

The cationic polymers according to the invention preferably have a molecular weight of 10,000 g/mol to 50,000,000 g/mol, in particular 50,000 g/mol to 5,000,000 g/mol, particularly preferably 75,000 g/mol to 1,000,000 g/mol.

Within the meaning of the invention, preferred agents include these cationic polymers described above in a quantity of 0.1% by weight to 20.0% by weight, particularly preferably 0.2% by weight to 10.0% by weight, very particularly preferably 0.5 to 5.0% by weight, in each case based on the weight of the agent.

The following cationic polymers are most preferably used according to the invention in the agents according to the invention when the cationic polymers with regard to the above-mentioned formulas (I) to (IV) meet one or more of the following features:
R¹ and R⁴ each mean a methyl group,
X¹ stands for an NH group,
X² stands for an NH group,
A¹ and A² independently stand for ethane-1,2-diyl or propane-1,3-diyl,
R², R³, R⁵, and R⁶ independently stand for methyl or ethyl, particularly preferably for methyl,
R⁷ stands for a (C₁₀ to C₂₄) alkyl group, in particular for decyl (caprinyl), dodecyl (lauryl), tetradecyl (myristyl), hexadecyl (cetyl), octadecyl (stearyl), eicosyl (arachyl), or docosyl (behenyl).

It is preferred according to the invention to select the structural unit of formula (III) from at least one structural unit of formulas (III-1) to (III-8)

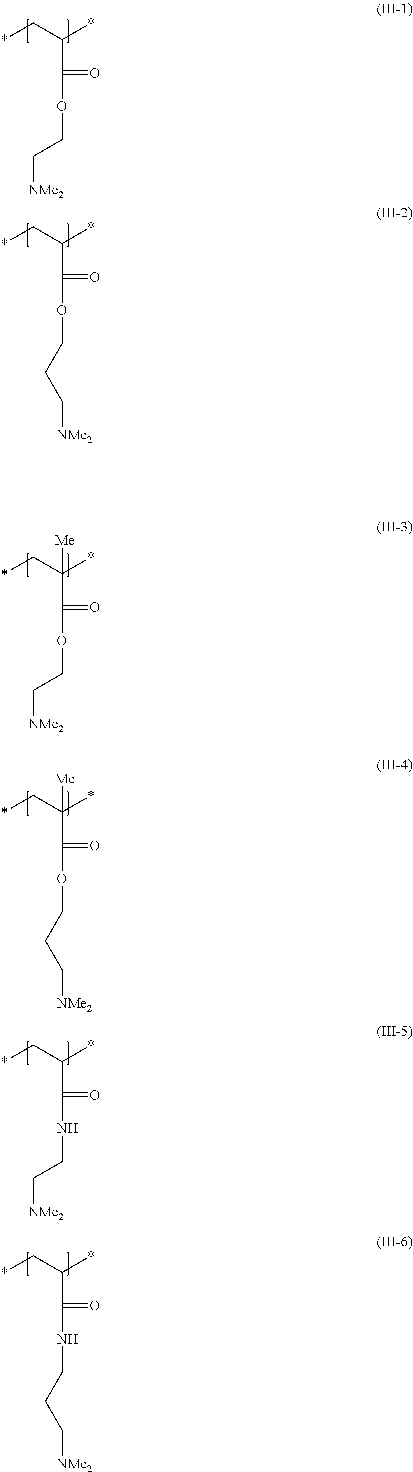

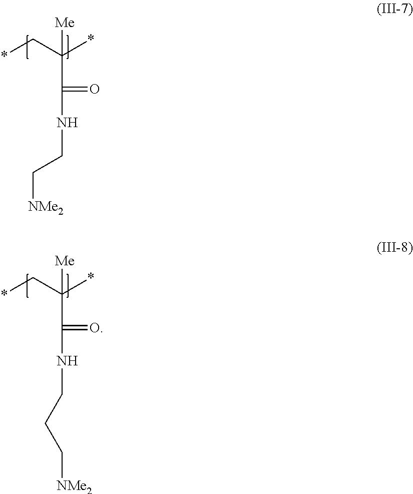

In addition, it has proven to be particularly preferable to select the structural unit(s) according to formula (III-7) and/or of formula (III-8) as the structural unit of formula (III). The structural unit of formula (III-8) is a very particularly preferred structural unit according to the invention,

Furthermore, with regard to achieving the object, it has proven to be preferable when the structural unit of formula (IV) is selected from at least one structural unit of formulas (IV-1) to (IV-8)

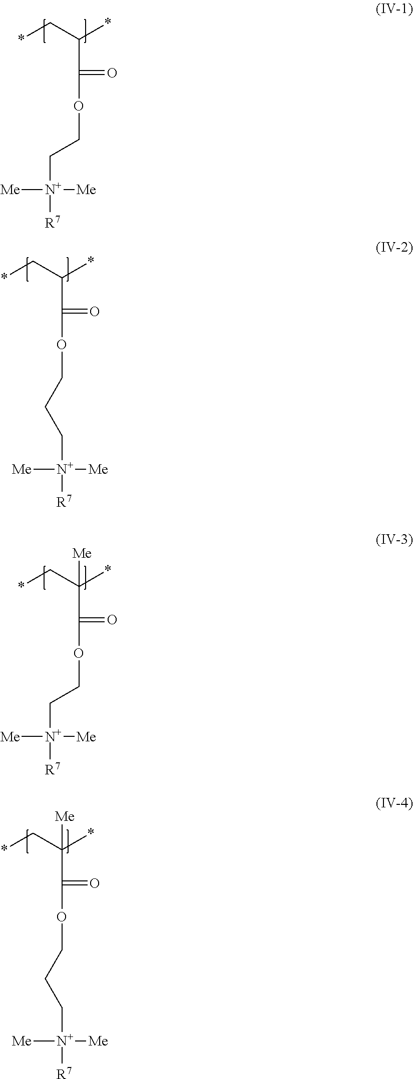

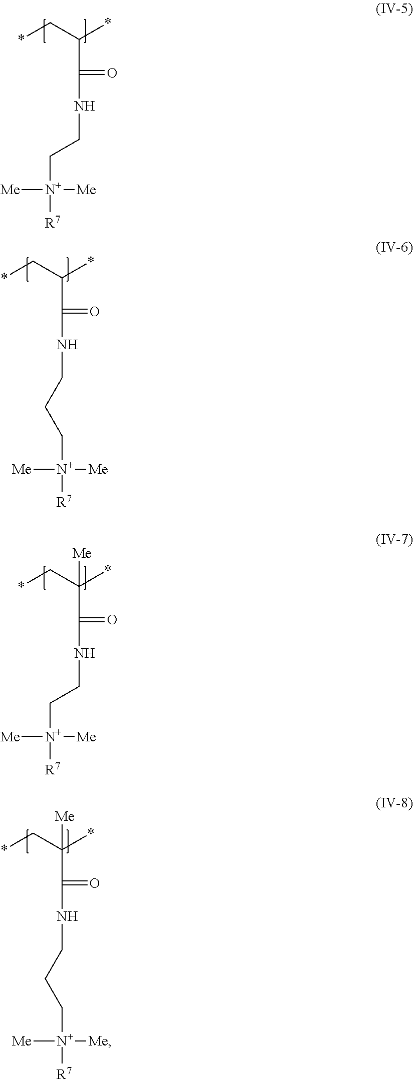

where R⁷ in each case stands for a (C₈ to C₃₀) alkyl group.

The structural unit(s) of formula (IV-7) and/or of formula (IV-8) are/is in turn particularly preferred structural unit(s) of formula (IV), wherein R⁷ in each case stands for octyl (capryl), decyl (caprinyl), dodecyl (lauryl), tetradecyl (myristyl), hexadecyl (cetyl), octadecyl (stearyl), eicosyl (arachyl), or docosyl (behenyl). According to the invention, the structural unit of formula (IV-8) represents a very particularly preferred structural unit of formula (IV).

A cationic polymer which is very particularly preferably included in the agent according to the invention includes at least one structural unit of formula (I), at least one structural unit of formula (II), at least one structural unit of formula (III-8), and at least one structural unit of formula (IV-8)

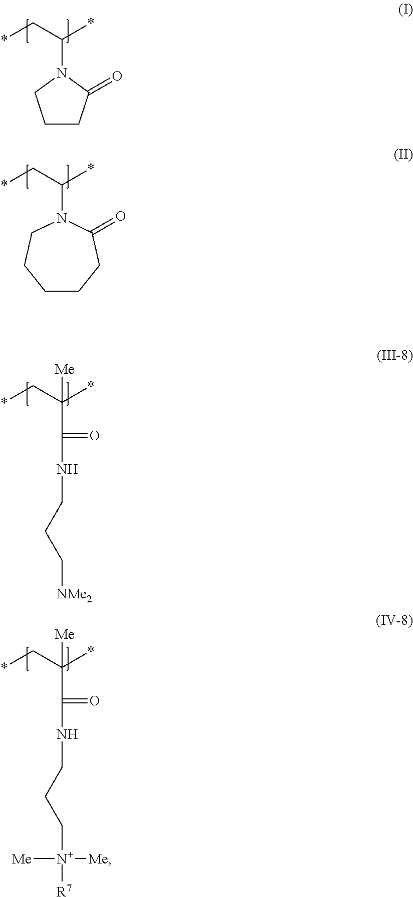

where R⁷ stands for octyl (capryl), decyl (caprinyl), dodecyl (lauryl), tetradecyl (myristyl), hexadecyl (cetyl), octadecyl (stearyl), eicosyl (arachyl), or docosyl (behenyl).

A very particularly preferred cationic polymer according to the invention is the copolymer of N-vinylpyrrolidone, N-vinylcaprolactam, N-(3-dimethylaminopropyl)methacrylamide, and 3-(methacryloylamino)propyl lauryl dimethylammonium chloride (INCI name: Polyquaternium-69), which is marketed, for example, by ISP under the trade name AquaStyle® 300 (28-32% by weight active substance in an ethanol-water mixture, molecular weight 350,000).

The polymers described thus far represent only a portion of polymers that are usable according to the invention. Without having to describe all the cationic and/or amphoteric polymers that are suitable according to the invention, in addition to their composition, the INCI declarations of the polymers that are preferred according to the invention are summarized. The polymers that are preferred according to the invention have the following INCI names: Polyquaternium-4, Polyquaternium-6, Polyquaternium-15, Polyquaternium-16, Polyquaternium-22, Polyquaternium-24, Polyquaternium-28, Polyquaternium-32, Polyquaternium-33, Polyquaternium-34, Polyquaternium-35, Polyquaternium-39, Polyquaternium-41, Polyquaternium-42, Polyquaternium-44, Polyquaternium-47, Polyquaternium-55, Polyquaternium-68, Polyquaternium-76, Polyquaternium-86, Polyquaternium-89, and Polyquaternium-95, and the mixtures thereof.

Further examples of preferred cationic polymers are the following:
cationized honey, for example the commercial product Honeyquat® 50,
polymeric dimethyldiallylammonium salts and copolymers thereof with esters and amides of acrylic acid and methacrylic acid. The products which are commercially available under the names Merquat® 100 (poly(dimethyldiallylammonium chloride)) and Merquat® 550 (dimethyldiallylammonium chloride-acrylamide copolymer) are examples of such cationic polymers having the INCI name Polyquaternium-7,
vinylpyrrolidone-vinylimidazolium methochloride copolymers, as marketed under the names Luviquat® FC 370, FC 550 and the INCI name Polyquaternium-16, and FC 905 and HM 552,
quaternized vinylpyrrolidone/dimethylaminoethyl methacrylate, for example vinylpyrrolidone/dimethylaminoethyl methacrylate methosulfate copolymer, which is marketed by Gaf Co., US under the trade names Gafquat® 755 N and Gafquat® 734 and the INCI name Polyquaternium-11,
quaternized polyvinyl alcohol,
polymers having quaternary nitrogen atoms in the main polymer chain, known under the names Polyquaternium-2, Polyquaternium-17, Polyquaternium-18, and Polyquaternium-27,
vinylpyrrolidone-vinylcaprolactam-acrylate terpolymers, which with acrylic acid esters and acrylic acid amides as the third monomer structural unit are marketed under the name Aquaflex® SF 40, for example.

The above-mentioned cationic polymers may be used individually or in any combinations with one another, in quantities of 0.01 to 10% by weight, preferably in quantities of 0.01 to 7.5% by weight, and very particularly preferably in quantities of 0.1 to 5.0% by weight. The very best results are obtained with quantities of 0.1 to 3.0% by weight, in each case based on the overall composition of the particular agent.

Amphoteric polymers according to the invention are polymers in which a cationic group is derived from at least one of the following monomers:

(i) monomers having quaternary ammonium groups of general formula (Mono1)

R¹—CH═CR²—CO—Z—(CₙH₂ₙ)—N⁽⁺⁾R²R³R⁴A⁽⁻⁾ (Mono1),
in which R¹ and R² independently stand for hydrogen or a methyl group, and R³, R⁴, and R⁵ independently stand for alkyl groups having 1 to 4 carbon atoms, Z is an NH group or an oxygen atom, n is an integer from 2 to 5, and A⁽⁻⁾ is the anion of an organic or inorganic acid,
   (ii) monomers having quaternary ammonium groups of general formula (Mono2)

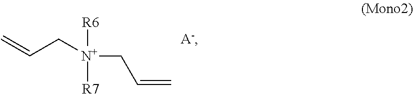

where R⁶ and R⁷ independently stand for a (C₁ to C₄) alkyl group, in particular for a methyl group, and
A⁻ is the anion of an organic or inorganic acid,
   (iii) monomeric carboxylic acids of general formula (Mono3)

R⁸—CH═CR⁹-0OOH (Mono3),
in which R⁸ and R⁹ independently stand for hydrogen or methyl groups.

Polymers are particularly preferred in which monomers of type (i) are used, in which R³, R⁴, and R⁵ are methyl groups, Z is an NH group, and A⁽⁻⁾ is a halide, methoxysulfate, or ethoxysulfate ion; acrylamidopropyltrimethylammonium chloride is a particularly preferred monomer (i). Acrylic acid is preferably used as monomer (ii) for the stated polymers.

Copolymers of at least one monomer (Mono1) or (Mono2) with monomer (Mono3), in particular copolymers of monomers (Mono2) and (Mono3), are particularly preferred amphoteric polymers. Copolymers of diallyldimethylammonium chloride and acrylic acid are amphoteric polymers which are very particularly preferably used according to the invention. These copolymers are marketed under the INCI name Polyquaternium-22 having the trade name Merquat® 280 (Nalco), among others.

Furthermore, in addition to a monomer (Mono1) or (Mono2) and a monomer (Mono3), the amphoteric polymers according to the invention may additionally include a monomer (Mono4):

(iv) Monomeric carboxylic acid amides of general formula (Mono4)

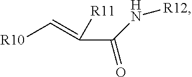

in which R¹⁰ and R¹¹ independently stand for hydrogen or methyl groups, and R¹² stands for a hydrogen atom or a (C₁ to C₈) alkyl group.

Amphoteric polymers which are based on a comonomer (Mono4) and very particularly preferably used according to the invention are terpolymers of diallyldimethylammonium chloride, acrylamide, and acrylic acid. These copolymers are marketed under the INCI name Polyquaternium-39 having the trade name Merquat® Plus 3330 (Nalco), among others.

The amphoteric polymers may generally be used directly and also in salt form, which is obtained by neutralizing the polymers with an alkali hydroxide, for example.

The above-mentioned cationic polymers may be used individually or in any combinations with one another, in quantities of 0.01 to 10% by weight, preferably in quantities of 0.01 to 7.5% by weight, and very particularly preferably in quantities of 0.1 to 5.0% by weight. The very best results are obtained with quantities of 0.1 to 3.0% by weight, in each case based on the overall composition of the particular agent.

As a further extremely preferred ingredient, a nonionic polymer is included in the compositions according to the invention. This polymer is selected from the nonionic polymers having at least one structural unit, selected from the group of structural units of formulas (M1) to (M3)

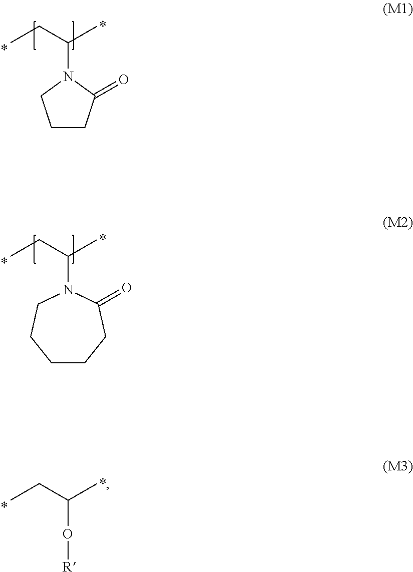

where R′ stands for a hydrogen atom or a (C₂ to C₁₈) acyl group.

According to the above formulas and all of the formulas below, a chemical bond denoted by the symbol “*” stands for a free valence of the corresponding structural fragment.

The properties of the composition according to the invention have proven to be particularly advantageous when it is provided as an aerosol spray, aerosol foam, pump spray, or pump foam. This preferred form of preparation is described in detail below.

According to the invention, a nonionic polymer is understood to mean a polymer which in a protic solvent at standard conditions bears essentially no structural unit having cationic or anionic groups which must be compensated for by counterions to maintain electroneutrality. Quaternized ammonium groups and protonated amines, for example, fall under cationic groups. Carboxylic acid groups and sulfonic acid groups, for example, fall under anionic groups.

The nonionic polymers as described above are preferably included in the compositions according to the invention in a quantity of 0.01% by weight to 10.0% by weight, particularly preferably 0.1% by weight to 10.0% by weight, very particularly preferably 0.1% by weight to 5.0% by weight, in each case based on the weight of the agent according to the invention.

Those nonionic polymers a) having at least one structural element of formula (M3) are preferably suited according to the invention which according to formula (M3) bear a hydrogen atom, an acetyl group, or a propanoyl group, in particular a hydrogen atom or an acetyl group, as R′.

The nonionic polymers according to the invention are in turn preferably selected from at least one polymer of the group comprising homopolymers and nonionic copolymers of N-vinylpyrrolidone; polyvinyl alcohol, and polyvinyl acetate.

Examples of suitable polyvinylpyrrolidones are commercial products such as Luviskol® K 90 or Luviskol® K 85 from BASF SE.

Suitable polyvinyl alcohols are marketed, for example, by Du Pont under the trade name Elvanol®, or by Air Products under the trade name Vinol® 523/540.

Suitable polyvinyl acetate is marketed, for example, by Air Products as an emulsion under the trade name Vinac®.

Compositions which as the nonionic polymer include at least one polymer selected from the group comprising polyvinylpyrrolidone and copolymers of N-vinylpyrrolidone and vinyl esters of carboxylic acids having 2 to 18 carbon atoms, in particular N-vinylpyrrolidone and vinyl acetate, are very particularly preferred according to the invention.

In addition, within the scope of this embodiment, in particular agents are very particularly preferred which in a cosmetically acceptable carrier include polyvinylpyrrolidone as the nonionic surfactant.

Furthermore, within the scope of this embodiment, in particular agents are very particularly preferred which in a cosmetically acceptable carrier include a copolymer which is produced from the monomers N-vinylpyrrolidone and vinyl acetate, in particular which is produced from no further monomer, as the nonionic polymer.

In turn, it is preferred when the molar ratio in the polymer of the structural units included from the monomer N-vinylpyrrolidone to the structural units included from the monomer vinyl acetate is in the range of 20:80 to 80:20, in particular 30:70 to 60:40.

Suitable copolymers of vinylpyrrolidone and vinyl acetate are obtainable, for example, from BASF SE under the trademark Luviskol® VA 37, Luviskol® VA 55, Luviskol® VA 64, and Luviskol® VA 73.

In addition to the nonionic polymers used, the agents according to the invention may include at least one further nonionic polymer that is different from the nonionic polymer described above.

Lastly, an extremely preferred optional ingredient of the compositions according to the invention is an amphoteric and/or zwitterionic surfactant. This surfactant class is customarily used in cleaning cosmetic compositions as a mild co-surfactant. These surfactants are typically not used in agents such as hair masks. An exception is hair mask foams, in which this surfactant class may be used as an auxiliary agent for foaming the compositions. In the compositions according to the invention, these ingredients may contribute greatly to improving the adherence of fragrance ingredients to the keratinic fiber.

Particularly suitable zwitterionic surfactants are the so-called betaines, such as N-alkyl-N,N-dimethylammonium glycinates, for example cocoalkyldimethylammonium glycinate, N-acyl-aminopropyl-N,N-dimethylammonium glycinate, for example cocoacylaminopropyldimethylammonium glycinate, and 2-alkyl-3-carboxymethyl-3-hydroxyethylimidazoline, in each case having 8 to 18 C atoms in the alkyl or acyl group, and cocoacylaminoethylhydroxyethylcarboxymethyl glycinate. A preferred zwitterionic surfactant is the fatty acid amide known under the INCI name Cocamidopropyl Betaine.

Ampholytic surfactants are understood to mean surface-active compounds which are capable of forming internal salts. Examples of suitable ampholytes are N-alkyl glycines, N-alkylpropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids, and alkylaminoacetic acids, in each case having approximately 8 to 24 C atoms in the alkyl group. Typical examples of amphoteric or zwitterionic surfactants are alkylbetaines, alkylamidobetaines, aminopropionates, aminoglycinates, imidazolinium betaines, and sulfobetaines.

Particularly preferred ampholytic surfactants are N-cocoalkylamino propionate, cocoacylaminoethylamino propionate, and C₁₂-C₁₈ acyl sarcosine. Coco betaine is a particularly preferred compound.

These ingredients are used in quantities of 0.01 to 8.0% by weight, based on the overall composition of the agent. Quantities of 0.05 to 7.0% by weight are preferred. Quantities of 0.1 to 6.0% by weight are particularly preferred, and quantities of 0.3 to 3.0% by weight are most preferred.

All ingredients that are customary in cosmetic compositions may also be added to this basic framework of ingredients. The selection of these components generally depends on the intended use of the hair treatment agents.

The compositions according to the invention may include further silicones In addition to the mandatory above-described silicones that include sugar structures. These optional silicones are preferably at least one silicone polymer selected from the group of dimethiconols and/or the group of amino functional silicones and/or the group of dimethicones and/or the group of cyclomethicones and/or the group of alkoxylated silicones.

The dimethicones according to the invention may be both linear and branched, as well as cyclic or cyclic and branched. Linear dimethicones may be represented by the following structural formula (Si1):

(SiR¹₃)—O—(SiR²₂—O—)ₓ—(SiR¹₃) (Si1)

Branched dimethicones may be represented by the structural formula (Si1.1):

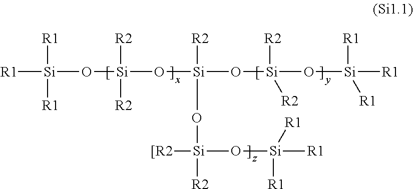

The radicals R¹ and R² each independently stand for hydrogen, a methyl radical, a C2 to C30 linear, saturated, or unsaturated hydrocarbon radical, a phenyl radical, and/or an aryl radical. The numbers x, y, and z are integers, and independently have a value from 0 to 50,000. The molecular weights of the dimethicones are between 1000 D and 10,000,000 D. The viscosities are between 100 and 10,000,000 cPs, measured at 25° C. using a glass capillary viscosimeter according to Dow Corning Corporate Test Method CTM 0004, Jul. 20, 1970. Preferred viscosities are between 1000 and 5,000,000 cPs, and very particularly preferred viscosities are between 10,000 and 3,000,000 cPs. The most preferred range is between 50,000 and 2,000,000 cPs. Viscosities around the range of approximately 60,000 cPs are extremely preferred. Reference is made to the product “Dow Corning 200 with 60,000 cSt” as an example.

Particularly preferred cosmetic or dermatological preparations according to the invention are characterized in that they include at least one silicone of formula (Si1.2)

(CH₃)₃Si—[O—Si(CH₃)₂]ₓ—O—Si(CH₃)₃ (Si1.2),

in which x stands for a number from 0 to 100, preferably 0 to 50, more preferably 0 to 20, and in particular 0 to 10.

The dimethicones (Si1) are included in the compositions according to the invention in quantities of 0.01 to 10% by weight, preferably 0.01 to 8% by weight, particularly preferably 0.1 to 7.5% by weight, and in particular 0.1 to 5% by weight, based on the overall composition.

Lastly, the silicone compounds are understood to include dimethiconols (Si8). The dimethiconols according to the invention may be both linear and branched, as well as cyclic or cyclic and branched. Linear dimethiconols may be represented by the following structural formula (Si8-I):

(SiOHR¹₂)—O—(SiR²₂—O—)ₓ—(SiOHR¹₂) (Si8-I)

Branched dimethiconols may be represented by the structural formula (Si8-II):

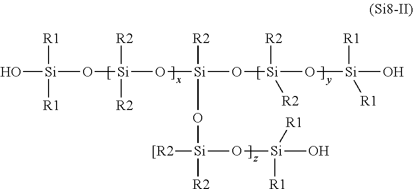

The radicals R¹ and R² independently stand for hydrogen, a methyl radical, a C2 to C30 linear, saturated, or unsaturated hydrocarbon radical, a phenyl radical, and/or an aryl radical. The numbers x, y, and z are integers, and independently have a value from 0 to 50,000. The molecular weights of the dimethicones are between 1000 D and 10,000,000 D. The viscosities are between 100 and 10,000,000 cPs, measured at 25° C. using a glass capillary viscosimeter according to Dow Corning Corporate Test Method CTM 0004, Jul. 20, 1970. Preferred viscosities are between 1000 and 5,000,000 cPs, and very particularly preferred viscosities are between 10,000 and 3,000,000 cPs. The most preferred range is between 50,000 and 2,000,000 cPs.

The following commercial products are mentioned as examples of these types of products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (the four latter-named products are GE Silicones), and Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, and Wacker-Belsil DM 60081 VP (all from Wacker-Chemie GmbH).

The dimethiconols (Si8) are included in the compositions according to the invention in quantities of 0.01 to 10% by weight, preferably 0.01 to 8% by weight, particularly preferably 0.1 to 7.5% by weight, and in particular 0.1 to 5% by weight of dimethiconol, based on the composition.

Particularly preferred agents according to the invention include one or more amino functional silicones. Such silicones may be described, for example, by the formula (Si-2):

M(RₐQ_{b}SiO_{(4-a-b)/2})ₓ(R_{c}SiO_{(4-c)/2})_{y}M (Si-2),

where in the above formula
R is a hydrocarbon or a hydrocarbon radical having 1 to approximately 6 carbon atoms,
Q is a polar radical of general formula —R¹HZ,
   where
   R¹ is a divalent connecting group which is bound to hydrogen and the radical Z, and composed of carbon and hydrogen atoms, carbon, hydrogen, and oxygen atoms, or carbon, hydrogen, and nitrogen atoms, and
   Z is an organic amino functional radical having at least one amino functional group;
a assumes values in the range of approximately 0 to approximately 2,
b assumes values in the range of approximately 1 to approximately 3,
a+b is less than or equal to 3,
c is a number in the range of approximately 1 to approximately 3,
x is a number in the range of 1 to approximately 2000, preferably from approximately 3 to approximately 50, and most preferably from approximately 3 to approximately 25,
y is a number in the range of approximately 20 to approximately 10,000, preferably from approximately 125 to approximately 10,000, and most preferably from approximately 150 to approximately 1.000, and
M is a suitable silicone end group as known in the prior art, preferably trimethylsiloxy.

Z according to formula (Si-2) is an organic amino functional radical having at least one functional amino group. One possible formula for Z is NH(CH₂)_{z}NH₂, where z is an integer greater than or equal to 1. Another possible formula for Z is —NH(CH₂)_{z}(CH₂)_{zz}NH, where both z and zz, independently from one another, are an integer greater than or equal to 1; this structure includes diamino ring structures such as piperazinyl. Z is most preferably an —NHCH₂CH₂NH₂ radical. Another possible formula for Z is —NH(CH₂)_{z}(CH₂)_{zz}NX₂ or —NX₂, where each X of X₂ is independently selected from the group comprising hydrogen and alkyl groups having 1 to 12 carbon atoms, and zz is 0.

Q according to formula (Si-2) is most preferably a polar amino functional radical of formula —CH₂CH₂CH₂NHCH₂CH₂NH₂.

In formula (Si-2), a assumes values in the range of 0 to 2, b assumes values in the range of 2 to 3, a+b is less than or equal to 3, and c is a number in the range of 1 to 3.

Cationic silicone oils such as the commercially available products Dow Corning (DC) 929 Emulsion, DC 2-2078, DC 5-7113, SM-2059 (General Electric), and SLM-55067 (Wacker) are suitable according to the invention.

Particularly preferred agents according to the invention are characterized in that they include at least one amino functional silicone of formula (Si3-a)

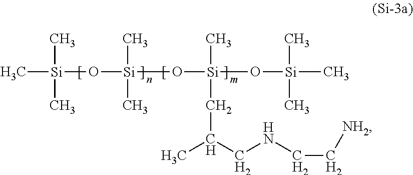

where m and n are numbers whose sum (m+n) is between 1 and 2000, preferably between 50 and 150, n preferably assuming values from 0 to 1999 and in particular from 49 to 149, and m preferably assuming values from 1 to 2000, in particular from 1 to 10.

These silicones are designated as Trimethylsilylamodimethicone according to the INCI declaration, and are obtainable, for example, under the name Q2-7224 (manufacturer: Dow Corning; a stabilized trimethylsilylamodimethicone).

Also particularly preferred are agents according to the invention which include at least one amino functional silicone of formula (Si-3b)

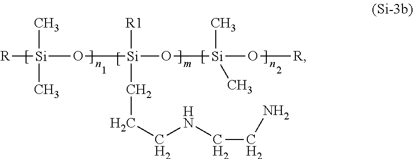

where
R stands for —OH, an (optionally ethoxylated and/or propoxylated) (C₁ to C₂₀) alkoxy group or a —CH₃ group,
R1 stands for —OH, a (C₁ to C₂₀) alkoxy group, or a —CH₃ group, and
m, n1, and n2 are numbers whose sum (m+n1+n2) is between 1 and 2000, preferably between 50 and 150, the sum (n1+n2) preferably assuming values from 0 to 1999 and in particular from 49 to 149, and m preferably assuming values from 1 to 2000, in particular from 1 to 10.

According to the INCI declaration, these silicones are designated as Amodimethicone or as functionalized Amodimethicone, for example Bis(C13-15 alkoxy) PG Amodimethicone (obtainable, for example, as the commercial product DC 8500 from Dow Corning), Trideceth-9 PG-Amodimethicone (obtainable, for example, as the commercial product Silcare Silicone SEA from Clariant).

Suitable diquaternary silicones are selected from compounds of general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c),

where the radicals R1 to R6 independently stand for C1 to C22 alkyl radicals which may include hydroxy groups, and wherein preferably at least one of the radicals includes at least 8 C atoms and the other radicals include 1 to 4 C atoms,
the radicals R7 to R12, independently of one another, are the same or different and mean C1 to C10 alkyl or phenyl,
A means a divalent organic connecting group,
n is a number from 0 to 200, preferably from 10 to 120, particularly preferably from 10 to 40, and X⁻ is an anion.

The divalent connecting group is preferably a C1 to C12 alkylene or alkoxyalkylene group, which may be substituted with one or more hydroxyl groups. The —(CH₂)₃—O—CH₂—CH(OH)—CH₂— group is particularly preferred.

The anion X⁻ may be a halide ion, an acetate, an organic carboxylate, or a compound of general formula RSO₃⁻, where R has the meaning of C1 to C4 alkyl radicals.

A preferred diquaternary silicone has the general formula (Si3d)

[RN⁺Me₂-A-(SiMe₂O)ₙ-SiMe₂-A-N⁺Me₂R]2CH₃COO⁻ (Si3d),

where A is the —(CH₂)₃—O—CH₂—CH(OH)—CH₂— group,
R is an alkyl radical having at least 8 C atoms, and
n is a number from 10 to 120.

Suitable silicone polymers having two terminal quaternary ammonium groups are known under the INCI name Quaternium-80. These are dimethylsiloxanes with two terminal trialkylammonium groups. Such diquaternary polydimethylsiloxanes are marketed by Evonik under the trade names Abil® Quat 3270, 3272, and 3474.

Hair treatment agents which are preferred according to the invention are characterized in that they include 0.01 to 10% by weight, preferably 0.01 to 8% by weight, particularly preferably 0.1 to 7.5% by weight, and in particular 0.2 to 5% by weight, of amino functional silicone(s) and/or diquaternary silicone, based on the weight of the hair treatment agents.

Further cationic silicone compounds having at least three terminal amino functional groups may likewise be used according to the invention. These cationic silicone polymers are characterized in that they have a silicone backbone structure and optionally a polyether portion and also at least one portion having an ammonium structure. Examples of preferred cationic silicone polymers within the meaning of the present invention are in particular the compounds having the INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22, and Silicone Quaternium-2 Panthenol Succinate and Silicone Quaternium-16/Glycidyl Dimethicone Crosspolymer. In particular Silicone Quaternium-22 is most preferred. This raw material is marketed, for example, by Evonik under the trade name Abil® T-Quat 60.

Another preferred cationic aminosilicone corresponds to the following formula:

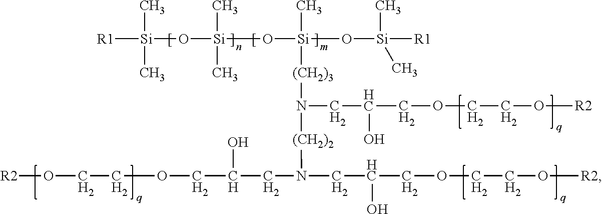

in which R1 stands for a methyl, ethyl, hydroxy, methoxy, or ethoxy group,
R2 stands for a straight-chain or branched C8 to C24 alkyl or alkylene radical, preferably a straight-chain or branched C9 to C22 alkyl or alkenyl radical, particularly preferably a straight-chain or branched C11 to C18 alkyl or alkenyl radical, most preferably a corresponding alkyl radical,
n and m each stand for integers from 1 to 1000, and q in each case stands for an integer from 0.2 to 50, preferably 4 to 30, particularly preferably 4 to 18, and most preferably 4 to 12.

The molecular weight of such compounds is 15,000 to 2,000,000, measured with a Brookfield RV rotational viscometer, spindle 5, at 20° C. The molecular weight is preferably 30,000 to 1,750,000 and particularly preferably 50,000 to 1,500,000. The nitrogen content of the silicones according to the invention is 0.03 to 4.2% by weight, preferably 0.1 to 2.8% by weight, and most preferably 0.16 to 1.4% by weight. Amino functional cationic silicones according to the invention of the above formula may be obtained from Clariant, for example. A product which is extremely preferred according to the invention is commercially available under the INCI names Trideceth-9-Amodimethicone and Trideceth-12.

Another particularly preferred amino functional silicone is at least one 4-morpholinomethyl-substituted silicone of formula (V)

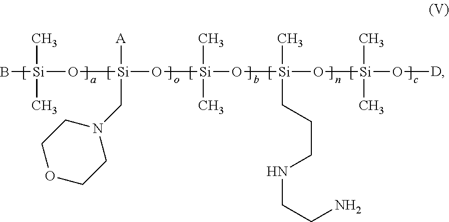

in which
A stands for a structural unit (I), (II), or (III)

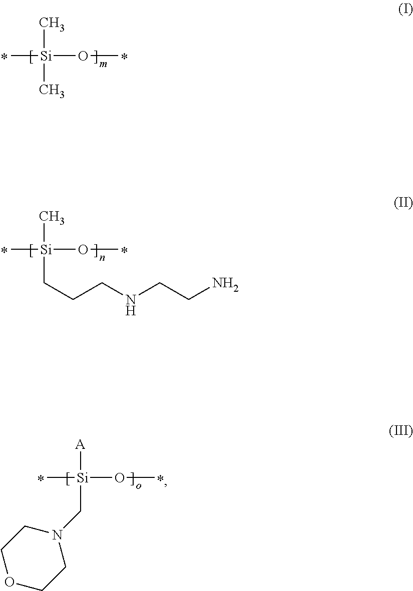

which is bound via an —O—,
or stands for an oligomeric or polymeric radical which is bound via an —O—, having structural units of formulas (I), (II), or (III), or one-half of an O atom to be connected to form a structural unit (III), or stands for —OH,
* stands for a bond to one of the structural units (I), (II), or (III) or for an end group B (Si-bound) or D (O-bound),
B stands for an —OH, —O—Si(CH₃)₃, —O—Si(CH₃)₂OH, or —O—Si(CH₃)₂OCH₃ group,
D stands for an —H, —Si(CH₃)₃, —Si(CH₃)₂OH, or —Si(CH₃)₂OCH₃ group,
a, b, and c stand for integers between 0 and 1000, with the condition that a+b+c>0,
m, n, and o stand for integers between 1 and 1000.

These types of amino functional silicones have the INCI name Amodimethicone/Morpholinomethyl Silsesquioxane Copolymer. A particularly suitable amodimethicone is the product having the trade name Wacker Belsil® ADM 8301E. It may be particularly advantageous according to the invention when solely the above-mentioned silicones are used as silicones.

These latter-named cationic amino functional silicone polymers are included in the compositions according to the invention in quantities of 0.01 to 5% by weight, preferably in quantities of 0.05 to 5% by weight, and very particularly preferably in quantities of 0.1 to 5% by weight. The very best results are obtained with quantities of 0.1 to 2.5% by weight, in each case based on the overall composition of the particular agent.

Polyammonium polysiloxane compounds are further silicone compounds according to the invention having amino functions. The polyammonium polysiloxane compounds may be obtained, for example, from GE Bayer Silicones under the trade name Baysilone®. The products having the names Baysilone TP 3911, SME 253, and SFE 839 are preferred. Use of Baysilone TP 3911 as the active component of the compositions according to the invention is very particularly preferred. The polyammonium polysiloxane compounds are used in the compositions according to the invention in a quantity of 0.01 to 10% by weight, preferably 0.01 to 7.5% by weight, particularly preferably 0.01 to 5.0% by weight, very particularly preferably 0.05 to 2.5% by weight, in each case based on the overall composition.

The cyclic dimethicones designated as Cyclomethicone according to INCI are also preferably usable according to the invention. Cosmetic or dermatological preparations according to the invention are preferred here which include at least one silicone of formula (Si-4)

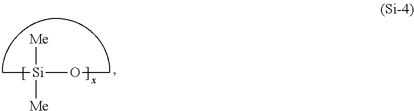

in which x stands for a number from 3 to 200, preferably from 3 to 10, more preferably from 3 to 7, and in particular 3, 4, 5, or 6.

Agents which are likewise preferred according to the invention are characterized in that they include at least one silicone of formula (Si-5)

R₃Si—[O—SiR₂]ₓ—(CH₂)ₙ—[O—SIR₂]_{y}—O—SiR₃ (Si-5),

in which R stands for the same or different radicals from the group H, phenyl, benzyl, and —CH₂—CH(CH₃)Ph, C₁₋₂₀ alkyl radicals, preferably —CH₃, —CH₂CH₃, —CH₂CH₂CH₃, —CH(CH₃)₂, CH₂CH₂CH₂CH₃, —CH₂CH(CH₃)₂, —CH(CH₃)CH₂CH₃, or —C(CH₃)₃, x and y each stand for a number from 0 to 200, preferably from 0 to 10, more preferably from 0 to 7, and in particular 0, 1, 2, 3, 4, 5, or 6, and n stands for a number from 0 to 10, preferably from 1 to 8, and in particular 2, 3, 4, 5, 6.

In addition to the dimethicones, dimethiconols, amodimethicones, and/or cyclomethicones according to the invention, water-soluble silicones may be included as further silicones in the compositions according to the invention.

Corresponding hydrophilic silicones are selected, for example, from the compounds of formula(s) (Si-6) and/or (Si-7). In particular, preferred water-soluble surfactants based on silicone are selected from the group of dimethicone copolyols, which are preferably alkoxylated, in particular polyethoxylated or polypropoxylated.

According to the invention, dimethicone copolyols are preferably understood to mean polyoxyalkylene-modified dimethylpolysiloxanes of general formulas (Si-6) or (Si-7):

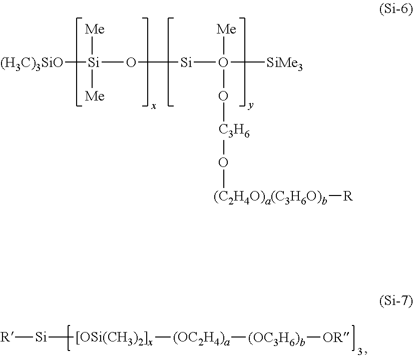

where the radical R stands for a hydrogen atom, an alkyl group having 1 to 12 C atoms, an alkoxy group having 1 to 12 C atoms, or a hydroxyl group, the radicals R′ and R″ mean alkyl groups having 1 to 12 C atoms, x stands for an integer from 1 to 100, preferably from 20 to 30, y stands for an integer from 1 to 20, preferably from 2 to 10, and a and b stand for integers from 0 to 50, preferably from 10 to 30.

Examples of particularly preferred dimethicone copolyols within the meaning of the invention are the products which are marketed by Union Carbide Corporation under the trade name Silwet, and by Dow Corning. Dow Corning 190 and Dow Corning 193 are dimethicone copolyols that are particularly preferred according to the invention.

The dimethicone copolyols are included in the compositions according to the invention in quantities of 0.01 to 10% by weight, preferably 0.01 to 8% by weight, particularly preferably 0.1 to 7.5% by weight, and in particular 0.1 to 5% by weight, of dimethicone copolyol, based on the composition.

Ester oils may particularly preferably be included as oil bodies in the active substance combination according to the invention. Ester oils are defined as follows:

Ester oils are understood to mean the esters of C₆-C₃₀ fatty acids with C₂-C₃₀ fatty alcohols. The monoesters of fatty acids with alcohols having 2 to 24 C atoms are preferred. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaostearic acid, arachidonic acid, gadoleic acid, behenic acid, and erucic acid and the technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinic alcohol, linolyl alcohol, linolenyl alcohol, eleostearyl alcohol, arachidyl alcohol, gadoleyl alcohol, behenyl alcohol, erucyl alcohol, and brassidyl alcohol, and the technical mixtures thereof. Particularly preferred according to the invention are isopropyl myristate (Rilanit® IPM), isononanoic acid-C16-18 alkyl ester (Cetiol® SN), 2-ethylhexyl palmitate (Cegesoft® 24), stearic acid 2-ethylhexyl ester (Cetiol® 868), cetyl oleate, glycerin tricaprylate, coco fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).

Of course, the ester oils may also be alkoxylated with ethylene oxide, propylene oxide, or mixtures of ethylene oxide and propylene oxide. The alkoxylation may be present on the fatty alcohol portion, on the fatty acid portion, or on both portions of the ester oils. However, it is preferred according to the invention that the fatty alcohol has initially been alkoxylated, and subsequently esterified with fatty acid. These compounds are illustrated in a general manner In formula (D4-II):

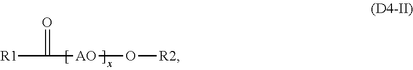

where R1 stands for a saturated or unsaturated, branched or unbranched, cyclic saturated or cyclic unsaturated acyl radical having 6 to 30 carbon atoms,
AO stands for ethylene oxide, propylene oxide, or butylene oxide,
X stands for a number between 1 and 200, preferably between 1 and 100, particularly preferably between 1 and 50, very particularly preferably between 1 and 20, extremely preferably between 1 and 10, and most preferably between 1 and 5,
R2 stands for a saturated or unsaturated, branched or unbranched, cyclic saturated or cyclic unsaturated alkyl, alkenyl, alkynyl, phenyl, or benzyl radical having 6 to 30 carbon atoms. Examples of fatty acid components used as radical R1 in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaostearic acid, arachidonic acid, gadoleic acid, behenic acid, and erucic acid, and the technical mixtures thereof. Examples of the fatty alcohol components as radical R2 in the ester oils are benzyl alcohol, isopropyl alcohol, caproic alcohol, caprylic alcohol, 2-ethylhexyl alcohol, caprin alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinic alcohol, linolyl alcohol, linolenyl alcohol, eleostearyl alcohol, arachidyl alcohol, gadoleyl alcohol, behenyl alcohol, erucyl alcohol, and brassidyl alcohol, and the technical mixtures thereof. An ester oil which is particularly preferred according to the invention is obtainable, for example, under the INCI name PPG-3 Benzyl Ether Myristate.

The following are also understood to mean ester oils:
dicarboxylic acid esters such as di-n-butyl adipate, di-(2-ethylhexyl) adipate, di-(2-ethylhexyl) succinate, and diisotridecyl acetate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate,
symmetrical, asymmetrical, or cyclic esters of carbonic acid with fatty alcohols, such as glycerin carbonate or dicaprylyl carbonate (Cetiol® CC),
trifatty acid esters of saturated and/or unsaturated linear and/or branched fatty acids with glycerin,
fatty acid partial glycerides, i.e., monoglycerides, diglycerides, and the technical mixtures thereof. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaostearic acid, arachidonic acid, gadoleic acid, behenic acid, and erucic acid, and the technical mixtures thereof. Oleic acid monoglycerides are preferably used.

The ester oils are used in the agents according to the invention in a quantity of 0.01 to 20% by weight, preferably 0.01 to 10.0% by weight, particularly preferably 0.01 to 7.5% by weight, most preferably 0.1 to 5.0% by weight. Of course, it is also possible according to the invention to use several ester oils at the same time.

Further oil bodies according to the invention are the following:
plant oils such as amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, black currant seed oil, jojoba oil, cocoa butter, linseed oil, macadamia nut oil, corn oil, almond oil, marula oil, evening primrose oil, olive oil, orange oil, palm oil, peach kernel oil, rapeseed oil, rice oil, sea buckthorn berry oil, sea buckthorn seed oil, sesame oil, shea butter, soy oil, sunflower oil, grapeseed oil, walnut oil, wheat germ oil, wild rose oil, and the liquid components of coconut oil.
animal oils, in particular triglyceride oils such as the liquid components of beef tallow and synthetic triglyceride oils.
liquid paraffin oils, isoparaffin oils, and synthetic hydrocarbons, as well as di-n-alkyl ethers having 12 to 36 C atoms total, in particular 12 to 24 C atoms, for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, as well as di-tert-butyl ether, diisopentyl ether, di-3-ethyldecyl ether, tert-butyl n-octyl ether, isopentyl n-octyl ether, and 2-methylpentyl n-octyl ether. The compounds 1,3-di-(2-ethylhexyl)cyclohexane (Cetiol® S) and di-n-octyl ether (Cetiol® OE) which are obtainable as commercial products may be preferred.

In many cases, the agents include at least one surface-active substance, in principle anionic as well as zwitterionic, ampholytic, nonionic, and cationic surface-active substances being suitable. The selection of the surface-active substances depends on the type of agent.

In preparations according to the invention, all anionic surface-active substances that are suitable for use on the human body are suited as anionic surfactants. Typical examples of anionic surfactants are the following:
linear and branched fatty acids having 8 to 30 C atoms (soaps),
ether carboxylic acids of formula R—O—(CH₂—CH₂O)ₓCH₂—COOH, in which R is a linear alkyl group having 8 to 30 C atoms, and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group, and sulfosuccinic acid monoalkylpolyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfofatty acid methyl esters of fatty acids having 8 to 30 C atoms,
alkyl sulfates and alkyl polyglycol ether sulfates of formula R—O(CH₂—CH₂O)ₓOSO₃H, in which R is a preferably linear alkyl group having 8 to 30 C atoms, and x=0 or 1 to 12,
hydroxysulfonates substantially corresponding to at least one of the two following formulas, or the mixtures and the salts thereof: CH₃—(CH₂)_{y}CHOH—(CH₂)ₚ(CH—SO₃M)-(CH₂)_{z}—CH₂—O—(CₙH₂ₙO)ₓ—H and/or CH₃—(OH₂)_{y}(CH—SO₃M)-(CH₂)ₚCHOH—(CH₂)_{z}CH₂—O—(CₙH₂ₙO)ₓ—H, where in both formulas y and z are 0 or integers from 1 to 18, p=0, 1, or 2, and the sum (y+z+p) is a number from 12 to 18, x is 0 or a number from 1 to 30, n is an integer from 2 to 4, M=H or alkali, in particular sodium, potassium, or lithium, an alkaline earth ion, in particular magnesium, calcium, or zinc, and/or an ammonium ion which may optionally be substituted, in particular mono-, di-, tri-, or tetraammonium ions with C1 to C4 alkyl, alkenyl, or aryl radicals,
sulfated hydroxyalkylpolyethylene and/or hydroxyalkylenpropylene glycol ethers of formula R¹—(CHOSO₃M)-CHR³—(OCHR⁴—CH₂)ₙ—OR², where R¹ stands for a linear alkyl radical having 1 to 24 C atoms, R² stands for a linear or branched, saturated alkyl radical having 1 to 24 C atoms, R³ stands for hydrogen or a linear alkyl radical having 1 to 24 C atoms, R⁴ stands for hydrogen or a methyl radical, and M stands for hydrogen, ammonium, alkylammonium, or alkanolammonium, where the alkyl and alkanol radicals each include 1 to 4 C atoms, or a metal atom selected from lithium, sodium, potassium, calcium, or magnesium, and n stands for a number in the range of 0 to 12, and in addition the total number of C atoms included in R¹ and R³ is 2 to 44,
sulfonates of unsaturated fatty acids having 8 to 24 C atoms and 1 to 6 double bonds,
esters of tartaric acid and citric acid with alcohols, which represent addition products of approximately 2-15 mol ethylene oxide and/or propylene oxide with fatty alcohols having 8 to 22 C atoms,
alkyl and/or alkenyl ether phosphates of formula

R¹(OCH₂CH₂)ₙ—O—(PO—OX)—OR₂,
in which R¹ preferably stands for an aliphatic hydrocarbon radical having 8 to 30 carbon atoms, R² stands for hydrogen, a (CH₂CH₂O)ₙR² radical, or X, n stands for numbers from 1 to 10, and X stands for hydrogen, an alkali metal or alkaline earth metal or NR³R⁴R⁵R⁶, where R³ to R⁶ independently stand for hydrogen or a C₁ to C₄ hydrocarbon radical,
sulfated fatty acid alkylene glycol esters of formula RCO(AIkO)ₙSO₃M,
in which RCO— stands for a linear or branched, aliphatic, saturated and/or unsaturated acyl radical having 6 to 22 C atoms, Alk stands for CH₂CH₂, CHCH₃CH₂, and/or CH₂CHCH₃, n stands for numbers from 0.5 to 5, and M stands for a metal such as an alkali metal, in particular sodium, potassium, or lithium, an alkaline earth metal, in particular magnesium, calcium, or zinc, or an ammonium ion such as ⁺NR³R⁴R⁵R⁶, where R³ to R⁶ independently stand for hydrogen or a C₁ to C₄-hydrocarbon radical,
monoglyceride sulfates and monoglyceride ether sulfates of formula

R⁸OC—(OCH₂CH₂)ₓOCH₂—[CHO(CH₂CH₂O)_{y}H]—CH₂O(CH₂CH₂O)_{z}—SO₃X,
in which R⁸CO stands for a linear or branched acyl radical having 6 to 22 carbon atoms, x, y, and z in sum stand for 0 or for numbers from 1 to 30, preferably 2 to 10, and X stands for an alkali metal or alkaline earth metal. Typical examples of monoglyceride (ether) sulfates which are suitable within the meaning of the invention are the reaction products of lauric acid monoglyceride, coco fatty acid monoglyceride, palmitic acid monoglyceride, stearic acid monoglyceride, oleic acid monoglyceride, and tallow fat acid monoglyceride, and the ethylene oxide addition products with sulfur trioxide or chlorosulfonic acid in the form of their sodium salts. Monoglyceride sulfates in which R⁹CO stands for a linear acyl radical having 8 to 18 carbon atoms are preferably used,
amide ether carboxylic acids, R¹—CO—NR²—CH₂CH₂—O—(CH₂CH₂O)ₙCH₂COOM, where R¹ stands for a straight-chain or branched alkyl or alkenyl radical having 2 to 30 carbon atoms in the chain, n stands for an integer from 1 to 20, and R² stands for hydrogen, a methyl, ethyl, propyl, isopropyl, n-butyl, t-butyl, or isobutyl radical, and M stands for hydrogen or a metal such as an alkali metal, in particular sodium, potassium, or lithium, or an alkaline earth metal, in particular magnesium, calcium, or zinc, or an ammonium ion such as ⁺NR³R⁴R⁵R⁶, where R³ to R⁶ independently stand for hydrogen or a C₁ to C₄ hydrocarbon radical. Such products are obtainable, for example, from Chem-Y under the product name Akypo®.
acylglutamates of formula XOOC—CH₂CH₂CH(C(NH)OR)—COOX, in which RCO stands for a linear or branched acyl radical having 6 to 22 carbon atoms and 0 and/or 1, 2, or 3 double bonds, and X stands for hydrogen, an alkali metal and/or alkaline earth metal, ammonium, alkylammonium, alkanolammonium, or glucammonium,
condensation products of a water-soluble salt of a water-soluble protein hydrolysate with a C8-C30 fatty acid. Such products have been commercially available for quite some time under the trademarks Lamepon®, Maypon®, Gluadin®, Hostapon® KCG, or Amisoft®,
alkyl and/or alkenyl oligoglycoside carboxylates, sulfates, phosphates, and/or isethionates,
acyl lactylates, and
hydroxy mixed ether sulfates.

If the mild anionic surfactants include polyglycol ether chains, it is very particularly preferred that they have a narrow homolog distribution. In addition, in the case of mild anionic surfactants having polyglycol ether units, it is preferred that the number of glycol ether groups is 1 to 20, preferably 2 to 15, particularly preferably 2 to 12. Particularly mild anionic surfactants having polyglycol ether groups without a narrow homolog distribution may also be obtained, for example, when on the one hand the number of polyglycol ether groups is 4 to 12, and on the other hand, Zn or Mg ions are selected as the counterion. The commercial product Texapon® ASV is an example of such.

Examples of nonionic surfactants are the following:
addition products of 2 to 50 mol ethylene oxide and/or 0 to 5 mol propylene oxide with linear and branched fatty alcohols having 6 to 30 C atoms, fatty alcohol polyglycol ethers, fatty alcohol polypropylene glycol ethers, or mixed fatty alcohol polyethers,
addition products of 2 to 50 mol ethylene oxide and/or 0 to 5 mol propylene oxide with linear and branched fatty alcohols having 6 to 30 C atoms, fatty acid polyglycol ethers, fatty acid polypropylene glycol ethers, or mixed fatty acid polyethers,
addition products of 2 to 50 mol ethylene oxide and/or 0 to 5 mol propylene oxide with linear and branched alkylphenols having 8 to 15 C atoms in the alkyl group, alkylphenol polyglycol ethers, alkylpolypropylene glycol ethers, or mixed alkylphenol polyethers,
addition products, closed with a methyl or C₂-C₆ alkyl radical end group, of 2 to 50 mol ethylene oxide and/or 0 to 5 mol propylene oxide with linear and branched fatty alcohols having 8 to 30 C atoms, with fatty acids having 8 to 30 C atoms, and with alkylphenols having 8 to 15 C atoms in the alkyl group, such as the types obtainable under the trade names Dehydol® LS and Dehydol® LT (Cognis),
C₁₂-C₃₀ fatty acid mono- and diesters of addition products of 1 to 30 mol ethylene oxide with glycerin,
addition products of 5 to 60 mol ethylene oxide with castor oil and hydrogenated castor oil,
polyol fatty acid esters, such as the commercial product Hydagen® HSP (Cognis) or Sovermol types (Cognis),
alkoxylated triglycerides,
alkoxylated fatty acid alkyl esters of formula (Tnio-1)

R¹CO—(OCH₂CHR²)_{w}OR³ (Tnio-1),
in which R¹CO stands for a linear or branched, saturated and/or unsaturated acyl radical having 6 to 22 carbon atoms, R² stands for hydrogen or methyl, R³ stands for linear or branched alkyl radicals having 1 to 4 carbon atoms, and w stands for numbers from 1 to 20,
aminoxides,
hydroxy mixed ethers, R¹O[CH₂CH(CH₃)O]ₓ(CH₂CHR²O)_{y}[CH₂CH(OH)R³]_{z}, where R¹ stands for a linear or branched, saturated or unsaturated alkyl and/or alkenyl radical having 2 to 30 C atoms, R² stands for hydrogen, a methyl, ethyl, propyl, or isopropyl radical, R³ stands for a linear or branched alkyl radical having 2 to 30 C atoms, x stands for 0 or a number from 1 to 20, y stands for a number from 1 to 30, and z stands for the number 1, 2, 3, 4, or 5,
sorbitan fatty acid esters and addition products of ethylene oxide with sorbitan fatty acid esters, for example polysorbates,
sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types,
sugar surfactants of the fatty acid-N-alkylpolyhydroxyalkylamide type,
fatty acid amide polyglycol ethers, fatty amine polyglycol ethers,
mixed ethers or mixed formals and polysorbates.

The surfactants are used in quantities of 0.05-45% by weight, preferably 0.1-30% by weight, and very particularly 0.5-25% by weight, based on the total agent used according to the invention.

Examples of emulsifiers that are usable according to the invention are the following:
addition products of 4 to 30 mol ethylene oxide and/or 0 to 5 mol propylene oxide with linear fatty alcohols having 8 to 22 C atoms, with fatty acids having 12 to 22 C atoms, and with alkylphenols having 8 to 15 C atoms in the alkyl group,
C₁₂-C₂₂ fatty acid mono- and diesters of addition products of 1 to 30 mol ethylene oxide with polyols having 3 to 6 carbon atoms, in particular with glycerin,
ethylene oxide and polyglycerol addition products with methyl glucoside fatty acid esters, fatty acid alkanolamides, and fatty acid glucamides,
C₈-C₂₂ alkyl mono- and oligoglycosides and the ethoxylated analogs thereof, with degrees of oligomerization of 1.1 to 5, in particular 1.2 to 2.0, and glucose as the sugar component being preferred,
mixtures of alkyl (oligo)glucosides and fatty alcohols, for example the commercially available product Montanov® 68,
addition products of 5 to 60 mol ethylene oxide with castor oil and hydrogenated castor oil,
partial esters of polyols having 3-6 carbon atoms with saturated fatty acids having 8 to 22 C atoms,
sterols from animal tissue (zoosterols, cholesterol, lanosterol) and from plant fats (phytosterols, ergosterol, stigmasterol, sitosterol), or from fungi and yeasts (mycosterols),
phospholipids (lecithins, phosphatidylcholines),
fatty acid esters of sugars and sugar alcohols, such as sorbitol,
polyglycerols and polyglycerol derivatives such as polyglycerol poly-12-hydroxy stearate (commercial product Dehymuls® PGPH).

The agents according to the invention preferably include the emulsifiers in quantities of 0.1-25% by weight, in particular 0.5-15% by weight, based on the overall agent.

The compositions according to the invention particularly preferably include fats as a further active substance. Fats are understood to mean fatty acids, fatty alcohols, natural and synthetic waxes, which may be present in solid form as well as in liquid form and in an aqueous dispersion, and natural and synthetic cosmetic oil components.

Linear and/or branched, saturated and/or unsaturated fatty acids having 6-30 carbon atoms may be used as fatty acids. Fatty acids having 10-22 carbon atoms are preferred. Mentioned as examples are isostearic acids, such as the commercial products Emersol® 871 and Emersol® 875, and isopalmitic acids such as the commercial product Edenor® IP 95, and all other fatty acids marketed under the trade name Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaostearic acid, arachidonic acid, gadoleic acid, behenic acid, and erucic acid, and the technical mixtures thereof. Fatty acid cuts obtainable from coconut oil or palm oil are typically particularly preferred; use of stearic acid in particular is generally preferred.

The quantity used is 0.1-15% by weight, based on the overall agent. The quantity is preferably 0.5-10% by weight, and quantities of 1-5% by weight may be very particularly preferred.

Saturated, singly or multiply unsaturated, branched or unbranched fatty alcohols having C₆-C₃₀ atoms, preferably C₁₀-C₂₂ atoms, and very particularly preferably C₁₂-C₂₂ carbon atoms may be used as fatty alcohols. Examples of alcohols which are usable within the meaning of the invention are decanol, octanol, octenol, dodecenol, decenol, octadienol, dodecadienol, decadienol, oleyl alcohol, erucyl alcohol, ricinol alcohol, stearyl alcohol, isostearyl alcohol, cetyl alcohol, lauryl alcohol, myristyl alcohol, arachidyl alcohol, capryl alcohol, caprin alcohol, linoleyl alcohol, linolenyl alcohol, and behenyl alcohol, and the guerbet alcohols thereof; this listing is by way of example and is not intended to be limiting. However, the fatty alcohols originate from preferably natural fatty acids, presumably typically being obtained from the esters of the fatty acids by reduction. Likewise usable according to the invention are fatty alcohol cuts which represent a mixture of different fatty alcohols. Such substances are commercially available, for example, under the names Stenol®, for example Stenol® 1618, or Lanette®, for example Lanette® O, or Lorol®, for example Lorol® C8, Lorol® C14, Lorol® C18, or Lorol® C8-18, HD-Ocenol®, Crodacol®, for example Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16, or Isocarb® 24. Of course, lanolin alcohols, which are commercially available under the names Corona®, White Swan®, Coronet®, or Fluilan®, for example, may also be used according to the invention. The fatty alcohols are used in quantities of 0.1-30% by weight, preferably in quantities of 0.1-20% by weight, based on the overall preparation.

Solid paraffin or isoparaffins, carnauba waxes, beeswaxes, candelilla waxes, ozokerites, ceresin, spermaceti, sunflower wax, fruit waxes such as apple wax or citrus wax, and micro waxes made of PE or PP may be used according to the invention as natural or synthetic waxes. These types of waxes are obtainable from Kahl & Co., Trittau, for example.

The quantity used is 0.1-50% by weight, based on the overall agent, preferably 0.1-20% by weight and particularly preferably 0.1-15% by weight, based on the overall agent.

The total quantity of oil and fat components in the agents according to the invention is usually 0.5-75% by weight, based on the overall agent. Quantities of 0.5-35% by weight are preferred according to the invention.

A further synergistic active substance according to the invention in the compositions according to the invention that include the active substance complex according to the invention are protein hydrolysates and/or the derivatives thereof.

Protein hydrolysates of plant, animal, or marine origin or of synthetic origin may be used according to the invention.

Examples of animal protein hydrolysates are elastin, collagen, keratin, silk, and milk protein hydrolysates, which may also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co.), Lexein® (Inolex), Sericin (Pentapharm), and Kerasol® (Croda).

Also preferred according to the invention are plant protein hydrolysates such as soy, almond, pea, moringa, potato, and wheat protein hydrolysates. Such products are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Serobiologiques.

Further protein hydrolysates which are preferred according to the invention are of marine origin. These include, for example, collagen hydrolysates from fish or algae, and protein hydrolysates from mussels or pearl hydrolysates. Examples of pearl extracts according to the invention are the commercial products Pearl Protein Extract BG® or Crodarom® Pearl.

Cationized protein hydrolysates are also included in the protein hydrolysates and the derivatives thereof, wherein the underlying protein hydrolysate may originate from animals, for example from collagen, milk, or keratin, from plants, for example from wheat, corn, rice, potatoes, soy, or almonds, from marine life forms, for example from fish collagen or algae, or from biotechnology-derived protein hydrolysates. Typical examples of the cationic protein hydrolysates and derivatives according to the invention are the commercially available products listed under the INCI names in the International Cosmetic Ingredient Dictionary and Handbook, (Seventh Edition 1997, The Cosmetic, Toiletry, and Fragrance Association, 1101 17th Street, N.W., Suite 300, Washington, D.C. 20036-4702).

The protein hydrolysates are included in the compositions in concentrations of 0.01% by weight to 20% by weight, preferably 0.05% by weight to 15% by weight, and very particularly preferably in quantities of 0.05% by weight to 5% by weight.

Another preferred group of ingredients in the compositions according to the invention that include the active substance complex according to the invention are vitamins, provitamins, or vitamin precursors.

Vitamins, provitamins, and vitamin precursors are preferred which are associated with the groups A, B, C, E, F, and H.

The group of substances denoted as vitamin A includes retinol (vitamin A₁) and 3,4-didehydroretinol (vitamin A₂). β-Carotene is the provitamin of retinol. For example, vitamin A acid and the esters thereof, vitamin A aldehyde, and vitamin A alcohol and the esters thereof, such as the palmitate and the acetate, are suitable as the vitamin A component. The agents according to the invention preferably include the vitamin A component in quantities of 0.05-1% by weight, based on the overall preparation.

The Vitamin B group or the Vitamin B complex includes the following, among others:

Vitamin B₁ (thiamin)
Vitamin B₂ (riboflavin)
Vitamin B₃. The compounds nicotinic acid and nicotinic acid amide (niacinamide) are frequently referred to by this name. Nicotinic acid amide, which is preferably included in the agents used according to the invention in quantities of 0.05 to 1% by weight, based on the overall agent, is preferred according to the invention.
Vitamin B5 (pantothenic acid, panthenol, and pantolactone). Within this group, panthenol and/or pantolactone is/are preferably used. Derivatives of panthenol which are usable according to the invention are in particular the esters and ethers of panthenol and cationically derivatized panthenols. Examples of individual representatives are panthenol triacetate, panthenol monoethyl ether and the monoacetate thereof, and cationic panthenol derivatives. Pantothenic acid is preferably used in the present invention as the derivative in the form of the more stable calcium salts and sodium salts (Ca pantothenate, Na pantothenate).
Vitamin B6 (pyridoxine as well as pyridoxamine and pyridoxal).

The stated compounds of the vitamin B type, in particular vitamins B₃, B₅, and B₆, are preferably included in the agents according to the invention in quantities of 0.05-10% by weight, based on the overall agent. Quantities of 0.1-5% by weight are particularly preferred.

Vitamin C (ascorbic acid). Vitamin C is preferably used in the agents according to the invention in quantities of 0.1 to 3% by weight, based on the overall agent. Use in the form of the palmitic acid ester, the glucosides, or phosphates may be preferred. Use in combination with tocopherols may likewise be preferred.

Vitamin E (tocopherols, in particular α-tocopherol). Tocopherol and its derivatives, which include in particular the esters such as the acetate, the nicotinate, the phosphate, and the succinate, are preferably included in the agents according to the invention in quantities of 0.05-1% by weight, based on the overall agent.

Vitamin F. The term “Vitamin F” is customarily understood as essential fatty acids, in particular linoleic acid, linolenic acid, and arachidonic acid.

Vitamin H. The compound (3aS,4S,6aR)-2-oxohexahydrothienol[3,4-d]imidazole-4-valeric acid is referred to as vitamin H, which has become known in common usage as biotin. Biotin is preferably included in the agents according to the invention in quantities of 0.0001 to 1.0% by weight, in particular in quantities of 0.001 to 0.01% by weight.

The compositions according to the invention preferably include vitamins, provitamins, and vitamin precursors from the groups A, B, E, and H. Panthenol, pantolactone, pyridoxine, and the derivatives thereof, as well as nicotinic acid amide and biotin, are particularly preferred.

A particularly preferred group of ingredients in the cosmetic compositions according to the invention are the following betaines: carnitine, carnitine tartrate, carnitine magnesium citrate, acetylcarnitine, betalains, 1,1-dimethylproline, choline, choline chloride, choline bitartrate, choline dihydrogen citrate, and the compound N,N,N-trimethylglycine, which is referred to in the literature as betaine.

Carnitine, histidine, choline, and betaine are preferably used. In one particularly preferred embodiment of the invention, L-carnitine tartrate is used as the active substance.

In another embodiment which is preferred according to the invention, the compositions according to the invention include bioquinones. In the agents according to the invention, one or more ubiquinone(s) and/or plastoquinone(s) is/are understood to be suitable bioquinones. Ubiquinones preferred according to the invention have the following formula:

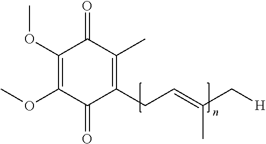

where n=6, 7, 8, 9, or 10.

Coenzyme Q-10 is most preferred.

Preferred compositions according to the invention include purine and/or purine derivatives in narrow quantity ranges. Here, cosmetic agents preferred according to the invention are characterized in that they contain, based on their weight, 0.001 to 2.5% by weight, preferably 0.0025 to 1% by weight, particularly preferably 0.005 to 0.5% by weight, and in particular 0.01 to 0.1% by weight of purine(s) and/or purine derivative(s). Cosmetic agents preferred according to the invention are characterized in that they include purine, adenine, guanine, uric acid, hypoxanthine, 6-purinethiol, 6-thioguanine, xanthine, caffeine, theobromine, or theophylline. Caffeine is most preferred in hair cosmetic preparations.

In another preferred embodiment of the present invention, the cosmetic agent includes ectoine ((S)-2-methyl-1,4,5,6-tetrahydro-4-pyrimidine carboxylic acid).

Agents are particularly preferred according to the invention which, based on their weight, include 0.00001 to 10.0% by weight, preferably 0.0001 to 5.0% by weight, and in particular 0.001 to 3% by weight of the active substances from the group comprising carnitine, coenzyme Q-10, ectoine, a vitamin of the B series, a purine and the derivatives thereof, or physiologically acceptable salts.

Taurine is a very particularly preferred care additive in the hair treatment agents according to the invention. Taurine is understood to mean solely 2-aminoethansulfonic acid, and a derivative is understood to mean the explicitly mentioned derivatives of taurine. The derivatives of taurine are understood to be N-monomethyltaurine, N,N-dimethyltaurine, taurine lysylate, taurine tartrate, taurine ornithate, lysyl taurine, and ornithyl taurine.

Agents according to the invention are particularly preferred which, based on their weight, include 0.0001 to 10.0% by weight, preferably 0.0005 to 5.0% by weight, particularly preferably 0.001 to 2.0% by weight, and in particular 0.001 to 1.0% by weight of taurine and/or a derivative of taurine.

The activity of the compositions according to the invention may be further increased by 2-pyrrolidinone-5-carboxylic acid and the derivatives (J) thereof. The sodium, potassium, calcium, magnesium, or ammonium salts, in which the ammonium ion bears one to three C₁ to C₄ alkyl groups in addition to hydrogen, are preferred. The sodium salt is very particularly preferred. The quantities used in the agents according to the invention are 0.05 to 10% by weight, particularly preferably 0.1 to 5% by weight, and in particular 0.1 to 3% by weight, based on the overall agent.

By the use of plant extracts as care substances, the hair treatment agents according to the invention may be formulated so that they are particularly close to nature, but still very effective in their care performance. It may even be possible to dispense with preservatives which are otherwise customary. Primarily preferred according to the invention are extracts of green tea, oak bark, nettle, witch hazel, hops, henna, chamomile, burdock root, horsetail, hawthorn, lime blossom, almond, aloe vera, spruce needle, horse chestnut, sandalwood, juniper berry, coconut, mango, apricot, lemon, wheat, kiwi fruit, melon, orange, grapefruit, sage, rosemary, birch, mallow, valerian, cuckoo flower, wild thyme, yarrow, thyme, lemon balm, restharrow, coltsfoot, marsh mallow, meristem, ginseng, coffee, cacao, moringa, ginger root, and Ayurvedic plant extracts such as Aegle marmelos (bilwa), Cyperus rotundus (nagarmotha), Emblica officinalis (amalaki), Morinda citrifolia (ashyuka), Tinospora cordifolia (guduchi), Santalum album (chandana), Crocus sativus (kumkuma), Cinnamomum zeylanicum, and Nelumbo nucifera (kamala), sweet grasses such as wheat, barley, rye, oats, spelt, corn, the various varieties of millet (proso millet, crabgrass, foxtail millet as examples), sugar cane, ryegrass, meadow foxtail, oatgrass, bentgrass, meadow fescue, moor grass, bamboo, cotton grass, fountain grasses, Andropogoneae (Imperata cylindrica, also referred to as flame grass or cogon grass), buffalo grass, cordgrasses, dog's tooth grasses, lovegrasses, Cymbopogon (lemongrass), Oryzeae (rice), Zizania (wild rice), beachgrass, perennial oatgrass, soft grasses, quaking grasses, poa grasses, wild rye, and Echinacea, in particular Echinacea purpurea (L.) Moench, all types of wine, and the pericarp of Litchi chinensis.

The plant extracts may be used according to the invention in pure or diluted form. If they are used in diluted form, they typically include approximately 2-80% by weight of active substance, and as solvent, the extraction agent or extraction agent mixture used in their production.

It may sometimes be necessary to use anionic polymers. Examples of anionic monomers of which such polymers may be composed are acrylic acid, methacrylic acid, crotonic acid, maleic acid anhydride, and 2-acrylamido-2-methylpropanesulfonic acid. The acid groups may be present in whole or in part as the sodium, potassium, ammonium, or mono- or triethanolammonium salt. Preferred monomers are 2-acrylamido-2-methylpropanesulfonic acid and acrylic acid.

Anionic polymers which include 2-acrylamido-2-methylpropanesulfonic acid alone or as a comonomer, wherein the sulfonic acid group may be present in whole or in part as the sodium, potassium, ammonium, or mono- or triethanolammonium salt, have proven to be very particularly effective.

The homopolymer of 2-acrylamido-2-methylpropanesulfonic acid, which is commercially available under the name Rheothik® 11-80, for example, is particularly preferred.

Preferred noniogenic monomers are acrylamide, methacrylamide, acrylic acid esters, methacrylic acid esters, vinylpyrrolidone, vinyl ethers, and vinyl esters.

Preferred anionic copolymers are acrylic acid-acrylamide copolymers and in particular polyacrylamide copolymers with monomers that include sulfonic acid groups. Such a polymer is included in the commercial product Sepigel® 305 from SEPPIC.

Likewise preferred anionic homopolymers are uncrosslinked and crosslinked polyacrylic acids. Allyl ethers of pentaerythritol, of sucrose, and of propylene may be preferred crosslinking agents. Such compounds are commercially available under the trademark Carbopol®, for example.

Copolymers of maleic acid anhydride and methyl vinyl ether, in particular those with crosslinking, are likewise color-preserving polymers. A maleic acid-methyl vinyl ether copolymer crosslinked with 1,9-decadiene is commercially available under the name Stabileze® QM.

The anionic polymers are preferably included in the agents according to the invention in quantities of 0.05 to 10% by weight, based on the overall agent. Quantities of 0.1 to 5% by weight are particularly preferred.

In another embodiment, the agents according to the invention may include further noniogenic polymers.

The following are examples of suitable further noniogenic polymers:
cellulose ethers such as hydroxypropyl cellulose, hydroxyethyl cellulose, and methyl hydroxypropyl cellulose, as marketed, for example, under the trademarks Culminal® and Benecel® (Aqualon) and Natrosol® types (Hercules).
starch and the derivatives thereof, in particular starch ethers such as Structure® XL (National Starch), a multifunctional salt-tolerant starch,
shellac.

The nonionic polymers are preferably included in the compositions according to the invention in quantities of 0.05 to 10% by weight, based on the overall agent. Quantities of 0.1 to 5% by weight are particularly preferred.

In another embodiment, the agents according to the invention should additionally include at least one UV light protection filter. UVB filters may be oil-soluble or water-soluble.

The following are mentioned as examples of oil-soluble substances:
3-benzylidene camphor, for example 3-(4-methylbenzylidene) camphor;
4-aminobenzoic acid derivatives, preferably 4-(dimethylamino)benzoic acid-2-ethylhexyl ester, 4-(dimethylamino)benzoic acid-2-octyl ester, and 4-(dimethylamino)benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, and 2-cyano-3-phenylcinnamic acid-2-ethylhexyl ester (octocrylene);
esters of salicylic acid, preferably salicylic acid-2-ethylhexyl ester, salicylic acid-4-isopropylbenzyl ester, and salicylic acid homomenthyl ester;
derivatives of benzophenone, preferably 2-hydroxy-4-methoxybenzophenone, 2-hydroxy-4-methoxy-4′-methylbenzophenone, and 2,2′-dihydroxy-4-methoxybenzophenone;
esters of benzalmalonic acid, preferably 4-methoxybenzalmalonic acid di-2-ethylhexyl ester;
triazine derivatives such as 2,4,6-trianilino-(p-carbo-2′-ethyl-1′-hexyloxy)-1,3,5-triazine and octyl triazone;
propane-1,3-diones such as 1-(4-tert-butylphenyl)-3-(4′-methoxyphenyl)propane-1,3-dione;
   The following are suitable water-soluble substances:
2-phenylbenzimidazole-5-sulfonic acid and the alkali, alkaline earth, ammonium, alkylammonium, alkanolammonium, and glucammonium salts thereof;
sulfonic acid derivatives of benzophenones, preferably 2-hydroxy-4-methoxybenzophenone-5-sulfonic acid and the salts thereof;
sulfonic acid derivatives of 3-benzylidene camphor, such as 4-(2-oxo-3-bornylidenemethyl)benzenesulfonic acid and 2-methyl-5-(2-oxo-3-bornylidene)sulfonic acid and the salts thereof.

In particular derivatives of benzoyl methane, such as 1-(4′-tert-butylphenyl)-3-(4′-methoxyphenyl)propane-1,3-dione or 1-phenyl-3-(4′-isopropylphenyl)propane-1,3-dione, are suitable as typical UV-A filters. The UV-A and UV-B filters may of course also be used in mixtures. In addition to the mentioned soluble substances, also suitable for this purpose are insoluble pigments, in particular finely dispersed metal oxides or salts, such as titanium dioxide, zinc oxide, iron oxide, aluminum oxide, cerium oxide, zirconium oxide, silicates (talc), barium sulfate, and zinc stearate. The particles should have an average diameter of less than 100 nm, preferably between 5 and 50 nm, and in particular between 15 and 30 nm. They may have a spherical shape, although particles having a shape that is ellipsoidal or otherwise different from a spherical shape may also be used.

In addition, the cosmetic agents may include further active substances, auxiliary substances, and additives such as the following:
structurizers such as maleic acid and lactic acid,
swelling agents such as urea, allantoin, carbonates, or hydantoin,
dimethyl isosorbide and cyclodextrins,
dyes for coloring the agent,
complexing agents such as EDTA, NTA, β-alaninediacetic acid, and phosphonic acids,
opacifiers such as latex, and styrene/PVP and styrene/acrylamide copolymers,
pearlescent agents such as ethylene glycol mono- and distearate and PEG-3 distearate,
pigments,
stabilizing agents for hydrogen peroxide and other oxidizing agents,
propellants such as propane-butane mixtures, N₂O, dimethyl ether, CO₂, and air,
antioxidants,
fragrance oils, odorants, and fragrance ingredients.

With regard to further optional components and the quantities of these components used, express reference is made to the relevant handbooks known to those skilled in the art.

A further subject matter of the invention therefore relates to a method for hair treatment, in which a hair treatment agent according to Claim 1 is applied to the hair, and rinsed from the hair after an exposure period.

The exposure period is preferably a few seconds to 100 minutes, particularly preferably 1 to 50 minutes, and very particularly preferably 1 to 30 minutes.

The invention also encompasses a method in which a cosmetic agent according to Claim 1 is applied to the hair and remains there. According to the invention, “remaining on the hair” is understood to mean that the agent is not rinsed from the hair immediately after being applied. Instead, in this case the agent remains on the hair for more than 100 minutes and as long as until the next hair washing.

The invention also encompasses use of the cosmetic composition according to the invention for improving the finish, enhancing the luster, improving the moisture balance, protecting from oxidative damage, preventing greasiness of the keratinic fibers, and increasing the wash resistance of dyed keratinic fibers.

The following examples are intended to explain the subject matter of the present invention without limiting same.

### Examples

All stated quantities are parts by weight unless noted otherwise. The following formulations were prepared using known production methods.

Care spray, also usable in the form of a foam and/or as a hair mask:

| | K1 | K2 | K3 | K4 | K5 | K6 | K7 | K8 | K9 | K10 | K11 |
|---|---|---|---|---|---|---|---|---|---|---|---|
| Polymer JR 400 | 0.2 | 0.2 | 0.2 | 0.2 | 0.2 | 0.2 | 0.2 | 0.2 | 0.2 | 0.2 | 0.2 |
| Armocare VGH 70 | 0.5 | 0.5 | 0.5 | 0.5 | 0.5 | 0.5 | 0.5 | 0.5 | 0.5 | 0.5 | 0.5 |
| Stearamidopropyl | 0.5 | 0.5 | 0.5 | 0.5 | 0.5 | 0.5 | 0.5 | 0.5 | 0.5 | 0.5 | 0.5 |
| Dimethylamine | | | | | | | | | | | |
| PVP/VA copolymer | 1.0 | 1.0 | 1.0 | 1.0 | 1.0 | 1.0 | 1.0 | 1.0 | 1.0 | 1.0 | 1.0 |
| 60/40 | | | | | | | | | | | |
| Laurdimonium | 1.0 | 1.0 | 1.0 | 1.0 | 1.0 | 1.0 | 1.0 | 1.0 | 1.0 | 1.0 | 1.0 |
| Hydroxypropyl | | | | | | | | | | | |
| Hydrolyzed Keratin | | | | | | | | | | | |
| Arginine | 0.1 | — | — | — | 0.1 | — | — | 0.1 | — | — | — |
| Glutamine | — | 0.1 | — | — | — | 0.1 | — | — | — | — | — |
| Carnitine | — | — | 0.1 | — | — | — | 0.1 | 0.1 | — | — | 0.1 |
| Taurine | — | — | — | 0.1 | 0.1 | 0.1 | 0.1 | 0.1 | — | 0.1 | — |
| Histidine | — | — | — | — | — | — | — | — | 0.1 | — | 0.1 |
| Lysine | — | — | — | — | — | — | — | — | — | 0.1 | — |
| Panthenol | 0.2 | 0.2 | 0.2 | 0.2 | 0.2 | 0.2 | 0.2 | 0.2 | 0.2 | 0.2 | 0.2 |
| Cetrimonium Chloride | 5.0 | 5.0 | 5.0 | 5.0 | 5.0 | 5.0 | 5.0 | 5.0 | 5.0 | 5.0 | 5.0 |
| Ceteareth-25 | 0.3 | 0.3 | 0.3 | 0.3 | 0.3 | 0.3 | 0.3 | 0.3 | 0.3 | 0.3 | 0.3 |
| Panax ginseng root | 0.5 | — | 0.5 | 0.5 | — | 0.5 | — | 0.5 | 0.5 | 0.5 | 0.5 |
| extract, arginine, acetyl | | | | | | | | | | | |
| tyrosine, Arctium majus | | | | | | | | | | | |
| root extract, hydrolyzed | | | | | | | | | | | |
| soy protein, | | | | | | | | | | | |
| Polyquaternium-11, | | | | | | | | | | | |
| PEG-12 Dimethicone, | | | | | | | | | | | |
| calcium panthothenate, | | | | | | | | | | | |
| zinc gluconate, | | | | | | | | | | | |
| niacinamide, ornithine | | | | | | | | | | | |
| HCI, citrulline, | | | | | | | | | | | |
| glucosamine HCI, biotin | | | | | | | | | | | |
| ProSina ® | — | 0.5 | 0.5 | — | 0.5 | 0.5 | 0.5 | — | 0.5 | 0.5 | 0.5 |
| Dow Corning 193 | 0.2 | 0.2 | 0.2 | 0.2 | 0.2 | 0.2 | 0.2 | 0.2 | 0.2 | 0.2 | 0.2 |
| PEG-8 PG-Coco- | 0.5 | 0.5 | 0.5 | 0.5 | 0.5 | 0.5 | 0.5 | 0.5 | 0.5 | 0.5 | 0.5 |
| Glucoside | | | | | | | | | | | |
| Dimethicone | | | | | | | | | | | |
| Coco Betaine | 0.5 | 0.5 | 0.5 | 0.5 | 0.5 | 0.5 | 0.5 | 0.5 | 0.5 | 0.5 | 0.5 |
| Water, preservative, | ad 100 | ad 100 | ad 100 | ad 100 | ad 100 | ad 100 | ad 100 | ad 100 | ad 100 | ad 100 | ad 100 |
| and optional | | | | | | | | | | | |
| fragrance oils | | | | | | | | | | | |

The pH values of all formulations were adjusted to 2 to 6.

For application as a foam, the formulation in question is either filled together with a propellant into an aerosol container, or is dispensed as a foam from a pump bottle having an appropriate pump attachment, such as an air foamer.

For application as a hair mask or cream, fatty alcohol(s) such as cetylstearyl alcohol and/or ethylene glycol distearate and/or glycerin monostearate is/are added to the above-mentioned formulations in quantities of 0.2 to 5.0% by weight.

Shampoo:

| | S1 | S2 | S3 | S4 | S5 | S6 | S7 | S8 | S9 | S10 | S11 |
|---|---|---|---|---|---|---|---|---|---|---|---|
| Texapon ® N70 | 15.0 | 15.0 | 15.0 | 15.0 | 15.0 | 15.0 | 15.0 | 15.0 | 15.0 | 15.0 | 15.0 |
| Arlypon ® F | 0.15 | 0.15 | 0.15 | 0.15 | 0.15 | 0.15 | 0.15 | 0.15 | 0.15 | 0.15 | 0.15 |
| Antil ® 141 | 0.15 | 0.15 | 0.15 | 0.15 | 0.15 | 0.15 | 0.15 | 0.15 | 0.15 | 0.15 | 0.15 |
| Disodium | 6.0 | 6.0 | 6.0 | 6.0 | 6.0 | 6.0 | 6.0 | 6.0 | 6.0 | 6.0 | 6.0 |
| Cocoamphodiacetate | | | | | | | | | | | |
| Polyquaternium-10 | 0.5 | 0.5 | 0.5 | 0.5 | 0.5 | 0.5 | 0.5 | 0.5 | 0.5 | 0.5 | 0.5 |
| Laurdimonium | 1.0 | 1.0 | 1.0 | 1.0 | 1.0 | 1.0 | 1.0 | 1.0 | 1.0 | 1.0 | 1.0 |
| Hydroxypropyl | | | | | | | | | | | |
| Hydrolyzed Keratin | | | | | | | | | | | |
| Arginine | 0.1 | — | — | — | 0.1 | — | — | 0.1 | — | — | — |
| Glutamine | | 0.1 | — | — | — | 0.1 | — | — | — | — | — |
| Carnitine | — | — | 0.1 | — | — | — | 0.1 | 0.1 | — | — | 0.1 |
| Taurine | — | — | — | 0.1 | 0.1 | 0.1 | 0.1 | 0.1 | — | 0.1 | — |
| Histidine | — | — | — | — | — | — | — | — | 0.1 | — | 0.1 |
| Lysine | — | — | — | — | — | — | — | — | — | 0.1 | — |
| Cetiol ®HE | 3.0 | 3.0 | 3.0 | 3.0 | 3.0 | 3.0 | 3.0 | 3.0 | 3.0 | 3.0 | 3.0 |
| Panthenol | 0.2 | 0.2 | 0.2 | 0.2 | 0.2 | 0.2 | 0.2 | 0.2 | 0.2 | 0.2 | 0.2 |
| Dow Corning ® 193 | 0.1 | 0.1 | 0.1 | 0.1 | 0.1 | 0.1 | 0.1 | 0.1 | 0.1 | 0.1 | 0.1 |
| PEG-8 PG-Coco- | 1.0 | 1.0 | 1.0 | 1.0 | 1.0 | 1.0 | 1.0 | 1.0 | 1.0 | 1.0 | 1.0 |
| Glucoside | | | | | | | | | | | |
| Dimethicone | | | | | | | | | | | |
| Panax ginseng root | 0.5 | — | 0.5 | 0.5 | — | 0.5 | — | 0.5 | 0.5 | 0.5 | 0.5 |
| extract, arginine, acetyl | | | | | | | | | | | |
| tyrosine, Arctium majus | | | | | | | | | | | |
| root extract, hydrolyzed | | | | | | | | | | | |
| soy protein, | | | | | | | | | | | |
| Polyquaternium-11, | | | | | | | | | | | |
| PEG-12 Dimethicone, | | | | | | | | | | | |
| calcium panthothenate, | | | | | | | | | | | |
| zinc gluconate, | | | | | | | | | | | |
| niacinamide, ornithine | | | | | | | | | | | |
| HCI, citrulline, | | | | | | | | | | | |
| glucosamine HCI, biotin | | | | | | | | | | | |
| ProSina ® | — | 0.5 | 0.5 | — | 0.5 | 0.5 | 0.5 | — | 0.5 | 0.5 | 0.5 |
| Cremophor ® HRE 60 | 0.5 | 0.5 | 0.5 | 0.5 | 0.5 | 0.5 | 0.5 | 0.5 | 0.5 | 0.5 | 0.5 |
| Water, preservative, | ad 100 | ad 100 | ad 100 | ad 100 | ad 100 | ad 100 | ad 100 | ad 100 | ad 100 | ad 100 | ad 100 |
| and optional | | | | | | | | | | | |
| fragrance oils | | | | | | | | | | | |

The pH values of all formulations were adjusted to 4.5 to 5.8.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition that includes in a cosmetically acceptable carrier, based on the weight of the overall composition,
a) at least one amino acid and/or at least one oligopeptide and/or at least one cationic protein hydrolysate in a total quantity of 0.01 to 10.0% by weight, and
b) at least one sugar structure-containing silicone of the following formula 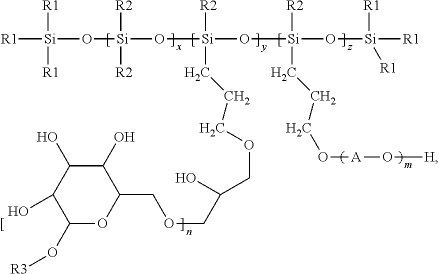
in which the radicals R1, R2, and R3 independently stand for a methyl, ethyl, propyl, isopropyl, hydroxy, methoxy, or ethoxy group, x, y, and z in each case stand for an integer from 1 to 1000, n and m each independently stand for an integer from 1 to 100, in a total quantity of 0.01 to 5.0% by weight.

2. The cosmetic composition according to claim 1, including the at least one amino acid, which is selected from the group consisting of alanine, arginine, asparagine, aspartic acid, cysteine, cystine, citrulline, glutamic acid, glutamine, glycine, histidine, hydroxylysine, hydroxyproline, isoleucine, leucine, lysine, methionine, phenylalanine, proline, serine, threonine, thyroxine, tryptophan, tyrosine, acetyl tyrosine, valine, betaine, ornithine, 1,1-dimethyl proline, hercynine, ergothioneine, and mixtures thereof.

3. The cosmetic composition according to claim 1, including the at least one oligopeptide having at least 3 and no more than 25 amino acids.

4. The cosmetic composition according to claim 3, wherein the oligopeptide includes the sequence Glu-Glu-Glu.

5. The cosmetic composition according to claim 1, including the at least one cationic protein hydrolysate, which is selected from the cationic keratin hydrolysates.

6. The cosmetic composition according to claim 1, further including at least one zwitterionic and/or amphoteric surfactant.

7. The cosmetic composition according to claim 6, wherein the zwitterionic and/or amphoteric surfactant are selected from the group consisting of cocamidopropyl betaine and coco betaine.

8. The cosmetic composition according to claim 1, further including at least one nonionic polymer that includes at least one structural unit selected from the group of structural units of formulas (M1) to (M3) 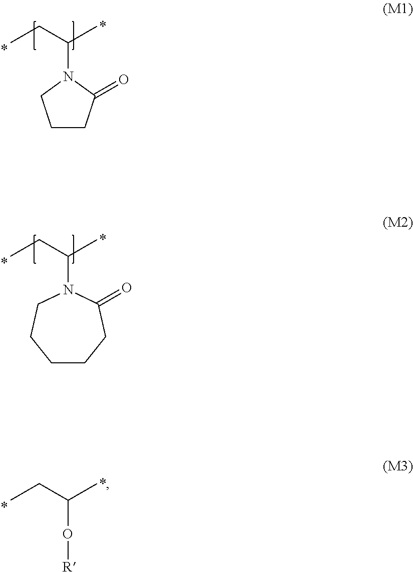
where R′ stands for a hydrogen atom or a (C₂ to C₁₈) acyl group, and the symbol “*” stands for a free valence of the corresponding structural fragment.

9. The cosmetic composition according to claim 1, including the cationic aminosilicone PEG-8 PG-Coco-Glucoside Dimethicone.

10. The cosmetic composition according to claim 1, further including at least one quaternary ammonium compound.

11. The cosmetic composition according to claim 10, wherein the quaternary ammonium compound is one or more compounds selected from the group consisting of stearamidopropyldimethylamine and/or distearoylethyl hydroxyethylmonium methosulfate, dicocoyl hydroxyethylmonium methosulfate, dipalmitoylethyl dimonium chloride, Quaternium-27, Quaternium-91, and behenoyl PG-trimonium chloride.

12. The cosmetic composition according to claim 1, further including at least one silicone selected from dimethicones, dimethiconols, cyclomethicones, amodimethicones, and dimethicone copolyols.

13. The cosmetic composition according to claim 1, further including at least one active substance selected from the group consisting of carnitine, taurine, coenzyme Q-10, ectoin, a purine and derivatives or physiologically acceptable salts thereof, and a vitamin of the B series.

14. A method for treating keratinic fibers, including:
applying a cosmetic composition according to claim 1 to the keratinic fibers, and rinsing the composition out of the keratinic fibers after an exposure period of a few seconds up to 45 minutes.
